(19) United States
(12) Patent Application Publication
(10) Pub. No.: US 2024/0254472 A1
(43) Pub. Date: Aug. 1, 2024

(54) COMPOSITIONS AND METHODS FOR SIMPLE SAMPLE EXTRACTION

(71) Applicant: Abbott Molecular Inc., Des Plaines, IL (US)

(72) Inventor: Gerard J. Gundling, Lake Forest, IL (US)

(21) Appl. No.: 18/289,431

(22) PCT Filed: May 5, 2022
(86) PCT No.: PCT/US22/27771

§ 371 Date: Nov. 3, 2023

Related U.S. Application Data
(60) Provisional application No. 63/184,857, filed on May 6, 2021.

(51) Int. Cl. C12N 15/10

(52) U.S. Cl. CPC C12N 15/1013; C12N 15/1017

(57) ABSTRACT

Compositions and methods for nucleic acid extraction and purification are provided.

This application claims priority to U.S. provisional patent application Ser. No. 63/184,857, filed May 6, 2021, which is incorporated herein by reference in its entirety.

### FIELD OF THE INVENTION

The invention relates, in part, to novel compositions and methods of nucleic acid extraction and purification.

### BACKGROUND

Most nucleic acid extraction systems follow certain basic steps. These systems must lyse biological samples and release the nucleic acids, bind the nucleic acids to some type of surface, remove contaminants, and elute the nucleic acids preferably in a more concentrated form. Some use solid matrices such as glass fibers or filters to bind the nucleic acids, others use magnetic particles. Some systems that do not require high levels of sensitivity may have more simple process but all must have a method that presents a nucleic acid target to an assay that can identify it. Pre-loaded cartridges are also used to simplify automation. With a pre-loaded system, the instrument does not need elaborate pumping and pipetting mechanisms to move the fluid. However, pre-loaded cartridges are complex devices in themselves, are costly to manufacture, and may still require steps such as mechanical mixing.

### SUMMARY

According to an aspect of the disclosure, a multi-layered composition for the extraction and isolation of nucleic acids from a biological sample is provided, the multi-layered composition including: a) a reaction vessel in which layers of the multi-layered composition are assembled; b) an uppermost layer within the reaction vessel including a concentrated semi-solid lysis paste; c) an intermediate layer including a low-temperature meltable wax; and d) a lower wash layer including an aqueous gel. In some aspects, the reaction vessel includes a tube of substantially circular cross-section, the tube having a top and a bottom. In some aspects, the top and the bottom of the tube are reversibly sealed. In certain aspects, the tube is J-shaped or U-shaped. In some aspects, the biological sample is a liquid biological sample. In some aspects, the liquid biological sample is a serum sample, a blood sample, a plasma sample, a saliva sample, or other type of biological sample. In some aspects, the low-temperature meltable wax is solid at room temperature. In some aspects, the low-temperature meltable wax begins to transition to liquid at a temperature above 50-55 degrees C. In certain aspects, the multi-layered composition further includes an adjacent layer including an elution buffer in fluid communication with the wash layer. In some aspects, the elution buffer is a low ionic strength elution buffer. In some aspects, the low ionic strength elution buffer is a phosphate buffer. In some aspects, the intermediate layer includes a plurality of low-temperature meltable wax layers wherein each of the plurality of low-temperature meltable wax layers is separated by an intervening sealing layer. In some aspects, the intervening sealing layer separating each of the plurality of low-temperature meltable wax layers includes a lower-temperature meltable wax that melts at a lower temperature than the layer immediately below it. In some aspects, the intervening sealing layer separating each of the plurality of low-temperature meltable wax layers includes mineral oil. In certain aspects, the intervening sealing layer separating each of the plurality of low-temperature meltable wax layers includes agarose. In some aspects, the plurality of low-temperature meltable wax layers are layered such that, in use, each of the plurality of low-temperature meltable wax layers melts sequentially from uppermost to lowermost. In some aspects, the intermediate layer further includes an internal control. In some aspects, the concentrated semi-solid lysis paste includes GITC, Tris-HCl, Tris base, and Tween-20. In some aspects, the concentrated semi-solid lysis paste has a residual moisture of less than 10% and does not flow. In some aspects, the concentrated semi-solid lysis paste has a residual moisture of less than 5%. In some aspects, the aqueous gel includes agarose or polyacrylamide. In some aspects, the uppermost layer of element b) further includes a plurality of metal oxide coated magnetic particles. In some aspects, the plurality of metal oxide coated particles are present in a quantity expected to represent a molar excess relative to the quantity of nucleic acids calculated to be present in the biological sample. In some aspects, the metal oxide coated particles are titanium oxide particles. In some aspects, the titanium oxide coated particles are copper titanium (CuTi) coated particles.

According to another aspect of the disclosure, a method for extracting and purifying nucleic acids from a biological sample is provided, the method including: a) providing a multi-layered composition for the extraction and isolation of nucleic acids from the biological sample, the multi-layered composition including: i) a reaction vessel in which layers of the multi-layered composition are assembled; ii) an uppermost layer within the reaction vessel including a concentrated semi-solid lysis paste; iii) an intermediate layer including a low-temperature meltable wax; and iv) a lower wash layer including an aqueous gel; b) layering a biological sample above the uppermost layer thereby forming solubilized lysis paste, wherein the solubilized lysis paste further includes a plurality of metal oxide coated magnetic particles; c) heating the reaction vessel to initiate melting of the intermediate layer resulting in density inversion characterized by the melting intermediate layer and rising through the solubilized concentrated semi-solid paste thereby mixing the biological sample, the solubilized concentrated semi-solid lysis paste and the plurality of metal oxide coated magnetic particles thereby resulting in the binding of nucleic acids within the biological sample to the metal oxide coated particles; c) positioning a magnet around or adjacent to the reaction vessel thereby attracting metal oxide coated particles and bound nucleic acids; and d) moving the magnet thereby driving the migration of attracted metal oxide coated particles and bound nucleic acids at least partially through the aqueous gel thereby removing extraction contaminants. In some aspects, the low-temperature meltable wax is solid at room temperature. In some aspects, the low-temperature meltable wax begins to transition to liquid at a temperature above 50-55 degrees C. In certain aspects, the multi-layered composition further includes an elution buffer in fluid communication with the wash layer, and the magnet is moved thereby driving the migration of attracted metal oxide coated particles and bound nucleic acids through the aqueous gel and into the elution buffer. In some aspects, the elution buffer is a low ionic strength elution buffer, optionally further wherein the low ionic strength elution buffer is a phosphate buffer. In some aspects, the intermediate layer of the multi-layered composition includes a plurality of low-temperature meltable wax layers wherein each of the plurality of low-temperature meltable wax layers is separated by an intervening sealing layer. In some aspects, the intervening sealing layer of the multi-layered composition separating each of the plurality of low-temperature meltable wax layers includes a lower-temperature meltable wax that melts at a lower temperature than the layer immediately below it. In other aspects, the intervening sealing layer of the multi-layered composition separating each of the plurality of low temperature meltable wax layers includes mineral oil. In some aspects, the intervening sealing layer of the multi-layered composition separating each of the plurality of low-temperature meltable wax layers includes agarose. In some aspects, the plurality of low-temperature meltable wax layers are layered such that, in use, each of the plurality of low-temperature meltable wax layers melts sequentially from uppermost to lowermost as the reaction vessel is heated. In some aspects, the plurality of metal oxide coated particles are titanium oxide particles. In some aspects, the titanium oxide particles are copper titanium (CuTi) coated particles. In certain aspects, the plurality of metal oxide coated particles are provided following the provision of the multi-layered composition of element a).

### BRIEF DESCRIPTION OF THE DRAWINGS

FIG. 1A-D presents schematic diagrams and photomicrographic images of wax integrity tests. FIG. 1A-C shows integrity tests using a wax mixture of 25% paraffin/75% Chill-out™ liquid wax. The left panel of FIG. 1A shows a schematic diagram of alternating wax and agarose layers as shown in the tubes in the right panel. The left panel of FIG. 1B shows a schematic diagram of agarose and wax layers as shown in the tubes in the right panel. The left panel of FIG. 1C shows a schematic diagram of wax and agarose layers as shown in the tubes in the right panel. FIG. 1D shows integrity tests using wax mixtures of microcrystalline wax with heavy mineral oil. The left panel shows a schematic diagram of agarose and wax layers as shown in the tubes in the right panel.

FIG. 2A-B presents a graphic representation and a schematic diagram of embodiments of the disclosure. FIG. 2A shows a graphic representation of an embodiment of the disclosure. Numerical labels indicate as follows: (1) extraction tube or reaction vessel; (2) concentrated semi-solid lysis paste (“lysis paste”); (3) low-temperature meltable wax layers of the intermediate layer; (4) intervening sealing layers of the intermediate layer; (5) agarose gel wash layer; (6) optional wax plug and/or optional cap. The gel wash layer removes contaminants and lysis reagent from the particles as they are magnetically drawn through that layer. The schematic diagram of FIG. 2B illustrates an embodiment of the disclosure wherein the intermediate layer comprises low-temperature meltable wax layers (25% paraffin/75% mineral oil; melting temperature 50-55° C.) separated by intervening sealing layers of lower-temperature meltable wax (5% paraffin/95% mineral oil; melting temperature 45-50° C.; “Wax IL”).

FIG. 3A-E presents a schematic diagram of a prepared extraction tube and graphs of real-time PCR (RT-PCR) results of HIV RNA and internal controls tested in samples extracted using compositions and methods of the disclosure. FIG. 3A shows a schematic diagram illustrating an embodiment of the disclosure. Inclusion of an HIV internal control within a low-temperature meltable wax layer is represented as “o”. FIG. 3B-C shows RT-PCR results of HIV RNA used in the two positive samples in the experiment. FIG. 3D-E shows RT-PCR results of the internal controls in all six samples used in the experiment.

FIG. 4A-G presents graphs and plots of real-time PCR (RT-PCR) results of HIV RNA and internal controls tested in samples extracted using compositions and methods of the disclosure. FIG. 4A-B shows RT-PCR results of HIV RNA used in the positive samples. FIG. 4C-D shows RT-PCR results of internal controls used in all experimental samples. FIG. 4E-G shows graphs of fit group analyses of the results of FIG. 4A-D. FIG. 4E shows a one-way analysis of HIV CT by sample. FIG. 4F shows a one-way analysis of IC CT by sample. FIG. 4G shows a one-way analysis of log cp/ml by sample.

FIG. 5A-C presents schematic diagrams of embodiments of the disclosure illustrated in Example 3 and graphs and plots of real-time PCR (RT-PCR) results of HIV RNA positive and negative controls and internal controls tested in samples extracted using compositions and methods of the disclosure. FIG. 5A shows schematic diagrams of embodiments of the disclosure, with intermediate layers comprising either five meltable wax layers (“five-layer”) or six meltable wax layers (“six-layer”). In both the five- and six-layer embodiments, the respective intermediate layers comprise low-temperature meltable wax layers (15% paraffin/85% mineral oil; “15% wax”) and the intervening sealing layers comprise lower-temperature meltable wax (5% paraffin/95% mineral oil; “5% wax IL”. FIG. 5B-C shows results of HIV RT-PCR reactions performed on samples extracted as described in Example 3. FIG. 5B shows results of target amplification (positive control samples). FIG. 5C shows results of internal control amplification in all samples.

### DETAILED DESCRIPTION

The present disclosure provides novel compositions and methods for extracting and isolating nucleic acids from biological samples. Aspects of the disclosure are based, in part, compositions and methods comprising simple reagents that can be easily assembled but embody a sophisticated design. A concentrated semi-solid lysis paste contains all the components for lysis and capture and relies on sample addition for re-hydration into a flowable liquid. Less-dense wax layers float to the surface of the dense lysate when melted and mix the lysis reaction by means of inverted density mixing without the need for mechanical mixing. An aqueous gel wash layer maintains its integrity throughout the process and prevents the lysate from contaminating the eluate. Magnetically captured particles are drawn through the gel, removing lysis contaminants without the need for additional washes, and may be drawn into an elution buffer. After elution, an eluate is easily added to an assay. Thus, embodiments of the disclosure greatly simplify nucleic acid extraction, using a minimal number of operational steps, and eliminating the need for complex operations, bulk reagents, dispensing pumps, and mechanical mixing during extraction. Furthermore, the wax seals the extraction tubes after cooling and eliminates liquid waste disposal.

In some aspects, compositions and methods of the disclosure combine extraction and purification steps such that they are performed simultaneously or in a fashion that reduces or eliminates traditional washes and mechanical steps. In some aspects, compositions and methods of the disclosure comprise a self-contained, heat-activated unit requiring only the addition of a liquid biological sample, heat, and magnetic movement of particles to perform nucleic acid extraction.

Aspects of compositions and methods of the disclosure build upon an earlier concept for extraction (U.S. Pat. No. 9,803,230, the disclosure of which is incorporated herein by reference), in which an aqueous gel was used to remove lysis buffer contaminants from magnetic particles as they were magnetically moved through the gel after capturing nucleic acids in a liquid lysis buffer. The movement through the gel “washed” the particles and removed the high levels of GITC-containing buffer from the particles. The particles were transferred to a buffer that then released the nucleic acids from the particles. However, that system as described required the separate addition of lysis buffer, sample, metal oxide coated magnetic particles, and elution buffer immediately prior to the extraction. Moreover, that method could not be pre-loaded because the high salt levels of the lysis buffer would diffuse into the gel, rendering the gel wash ineffective. In contrast, aspects of the disclosure use a concentrated semi-solid lysis paste, metal oxide coated magnetic particles, and a low ionic strength elution buffer, and most significantly, compartmentalize the extraction components by means of meltable wax layers within a single tube. The wax layers not only separate the lysis paste from the aqueous layers, but also mix the lysis reaction when melted during lysis incubation. In embodiments of the disclosure, the metal oxide coated magnetic particles are titanium oxide particles. In some embodiments, the titanium oxide particles are copper titanium (CuTi) coated particles (U.S. Pat. No. 10,392,613, the disclosure of which is incorporated herein by reference)

In some aspects, compositions and methods of the disclosure have several advantages over conventional silica extraction methods. One of skill in the art will recognize that one advantage is simplicity of operation, which in some embodiments comprises adding a sample to a pre-loaded extraction tube, heating the tube, collecting the magnetic particles on the side of the extraction tube, and moving them into an elution buffer chamber. In some embodiments, no pipetting of reagents is necessary. The lysate does not need to be mechanically mixed during extraction. Extraction tubes are also easily manufactured, requiring only the dispensing of gel, wax layers, and lysis paste into extraction tubes, rather than elaborate commodity manufacturing. In some embodiments, a minimum of reagents and plasticware are used, and plastic and liquid waste are greatly reduced. One of skill in the art with recognize that the reduction of steps also speeds up the extraction process. These advantages greatly reduce the instrumentation needed for automation. Compositions and methods of the disclosure represent a substantial improvement to existing nucleic extraction processes, and may be important for point-of-care testing and high-throughput processing of samples, for purposes including but not limited to blood banking and transplantation.

In aspects, the present disclosure relates to a multi-layered composition for the extraction and isolation of nucleic acids from a biological sample. As used herein, the term “biological sample” refers to samples obtained from a subject, from cells, tissues, or other biological sources. Biological samples may be naturally occurring, may be concentrates or suspensions of cells or tissues or fragments thereof in a buffer, may be products of cells or tissues, or may be synthetic nucleic acids. Non-limiting examples of biological samples include blood, bone marrow, tissue, surgical specimen, biopsy specimen, liquid biopsy specimen, tissue explant, organ culture, or any other tissue or cell preparation, or fraction or derivative thereof or isolated therefrom, etc. In aspects of the present disclosure biological samples comprise or are prepared to comprise residual moisture. For example, though not intended to be limiting, a sample having 0.2 ml of liquid will solubilize 1 g of lysis paste at 55° C. while a sample having 0.3 ml of liquid will solubilize 1 g of lysis paste at room temperature.

In aspects of the present disclosure, the biological sample is a liquid biological sample. Non-limiting examples of liquid biological samples include whole blood, serum, plasma, lymph, vitreous humor, aqueous humor, mucous, cerebrospinal fluid, saliva, urine, milk, ascites fluid, synovial fluid, peritoneal fluid, amniotic fluid, fermentation broths, cell culture products, nucleic acid synthesis products, or other biological fluid, etc. In embodiments of the present disclosure, nucleic acids may be obtained from any biological sample including, for example, primary cells, cell lines, freshly isolated cells or tissues, frozen cells or tissues, paraffin-embedded cells or tissues, fixed cells or tissues, and/or laser dissected cells or tissues. In some embodiments, a sample from which nucleic acids are isolated for use in methods of the invention is a control sample. Nucleic acids may be isolated from a subject, cell, or other source according to methods known in the art. Persons having skill in the art will understand how to obtain and prepare biological samples and liquid biological samples using art-known methods, including but not limited to, preparing plasma from blood, isolating cells from biological fluids, homogenizing tissue, disrupting cells or viral particles, preparing liquids from solid materials, diluting viscous fluids, filtering liquids, distilling liquids, concentrating liquids, inactivating interfering components, adding reagents, purifying nucleic acids, and the like.

As used herein, the term “subject” may refer to human or non-human animals, including mammals and non-mammals, vertebrates and invertebrates, and may also refer to any multicellular organism or single-celled organism such as a eukaryotic (including plants and algae) or prokaryotic organism, archaeon, microorganisms (e.g., bacteria, archaea, fungi, protists, viruses), and aquatic plankton. A subject may be considered to be a normal subject or may be a subject known to have or suspected of having a disorder, disease, or condition. Non-limiting examples of diseases or conditions include infectious diseases, such as human immunodeficiency virus (HIV), and Hepatitis A, B, C, D, and E viruses; monogenic disorders, such as sickle cell anemia, hemophilia, cystic fibrosis, Tay Sachs disease, Huntington's disease, and fragile X syndrome; chromosomal disorders, such as Down syndrome and Turner syndrome; polygenic disorders such as Alzheimer's disease, heart disease, diabetes, etc.; structural disorders such as deletions, insertions, and repeat expansions; and cancers.

Cells, tissues, or other sources or samples may include a single cell, a variety of cells, or organelles. It will be understood that a cell sample comprises a plurality of cells. As used herein, the term “plurality” means more than one. In some instances, a plurality of cells is at least 1, 10, 100, 1,000, 10,000, 100,000, 500,000, 1,000,000, 5,000,000, or more cells. A plurality of cells from which nucleic acids are isolated for use in compositions and methods of the disclosure may be a population of cells. A plurality of cells may include cells that are of the same cell type. In some embodiments, a cell from which nucleic acids are isolated for use in methods of the disclosure is a healthy normal cell, which is not known to have a disease, disorder, or abnormal condition. In some embodiments, a plurality of cells from which nucleic acids are isolated for use in methods of the disclosure includes cells having a known or suspected disease or condition or other abnormality, for example, a cell obtained from a subject diagnosed as having a disorder, disease, or condition, including, but not limited to a cell infected with a virus, a degenerative cell, a neurological disease-bearing cell, a cell model of a disease or condition, an injured cell, etc. In some embodiments, a cell is an abnormal cell obtained from cell culture, a cell line known to include a disorder, disease, or condition, including the non-limiting examples of disorders, disease, or conditions described elsewhere herein. In some embodiments of the invention, a plurality of cells is a mixed population of cells, meaning all cells are not of the same cell type. In some embodiments, a cell from which nucleic acids are isolated for use in methods of the invention is a control cell.

The multi-layered composition of the subject disclosure is formed by the sequential assembly of layers in a container or reaction vessel (also referred to elsewhere herein as an “experimental tube”). The material of which the reaction vessel is made is not critical so long as the material in no way interferes with aspects of the disclosed method for extraction and isolation of nucleic acids from a biological sample. For example, as will be discussed below, a magnetic field is utilized for the purpose of attracting magnetic particles. Given the importance of the magnetic field, the use of magnetic metals should be avoided. This requirement does not preclude all metals as, for example, austenite stainless steel structures will not be magnetic. Stainless steel having a ferrite or martensite structure will be magnetic and should be avoided. Glass and polymer formulations (e.g., polystyrene and polyethylene) are preferred for use in the formation of a reaction vessel.

The reaction vessel may comprise a tube of substantially circular cross-section, having a top and a bottom. A substantially circular cross-section is preferred (but not required) based on typical stock availability and prevalence of such cross-sections among materials used and consumed in the chemical and life-sciences industries. Non-limiting examples of tubes that may be used include but are not limited to 5 ml test tubes, 5 ml pipette tips, 1 ml pipette tips, or reaction vessels of custom design for use in instruments. In some embodiments of the disclosure, the top and the bottom of the tube are reversibly sealed. In some embodiments, only the top or the bottom of the tube is reversibly sealed. Non-limiting examples of materials that may be used to seal the top and bottom of the tube include a meltable hydrophobic wax, a meltable polymerizable material, or a removable plastic tip. In some embodiments, the top and/or bottom of the tube may be irreversibly sealed by a puncturable seal. One of skill in the art will understand how to choose a type of seal appropriate for a particular set of working conditions.

In some embodiments, multi-layered compositions of the disclosure may be manually layered within a reaction vessel. In other embodiments, a reaction vessel may be manufactured for disposable use with an automated molecular diagnostics analysis instrument. Reaction vessel size may vary in terms of volume, length, and configuration depending on how an extraction is to be performed (for example, but not limited to, in a manual benchtop format, with an automated analysis instrument, or a combination thereof) and initial sample volume. As a non-limiting example, a reaction vessel for manual benchtop use may have an overall volume of at least 1 ml, 2 ml, 5 ml, 10 ml, or more. A reaction vessel for use with an automated analytical instrument may have a smaller volume and/or length, for example but not limited to at least 0.25 ml, 0.5 ml, 1 ml, or more. In certain embodiments, the tube may be vertically oriented, such that the top and bottom openings are directly aligned. In some embodiments, a portion of the tube may be configured to comprise a bend, in order to maximize efficiency in an automated analysis instrument. In some embodiments, the tube is configured such that the overall shape of the tube resembles a “J” or a “U”. Such alternatives to a straight, vertically-oriented reaction vessel will be discussed in greater detail in connection with methods wherein metal oxide coated magnetic particles and bound nucleic acids are drawn through the gel wash layer in a non-vertically oriented portion of a reaction vessel.

While the disclosed composition and methods can be practiced manually in a benchtop format, in instances, the reaction vessel will be selected for use specifically with an automated analytical instrument. For example, the Abbott Alinity m (Abbott, Abbott Park, Ill.) is a fully integrated and automated molecular diagnostics analysis instrument with application, for example to polymerase chain reaction assays.

For discussion purposes, consider a multi-layered composition of the present disclosure wherein the reaction vessel is a polyethylene pipet having an inner diameter of approximately 20 mm. For purposes of the present disclosure, the reaction vessel will be vertically-oriented with a top and a bottom opening. The formation of the multi-layered composition will include pouring layers into the reaction vessel from the top. The bottom, therefore, will be reversibly sealed.

In the tubular instance under discussion, the bottom-most layer (i.e., the first layer to be poured) is a wash layer comprising an aqueous gel. In some embodiments, the aqueous gel is comprised of agarose or polyacrylamide. The aqueous gel may comprise at least 0.4%, 0.5%, 0.6%, 0.7%, 0.75%, 0.8%, 0.9%, 1%, or 1.5% (w/v) agarose or at least 0.5%, 1%, 5%, or 15% (w/v) polyacrylamide. One of skill in the art will understand that the aqueous gel must be of a concentration sufficient to support the sample, lysis, and wax layers but still allow the particles to be drawn through the gel during the extraction process. The aqueous gel may be prepared using an aqueous buffer compatible with downstream nucleic acids analyses to be performed on nucleic acids isolated using compositions and methods of the disclosure. As a non-limiting example, if the isolated nucleic acids will be analyzed using an Alinity m instrument, the aqueous gel may be prepared using System Diluent (Abbott, Abbott Park, Ill.) or other low salt non-phosphate containing buffer systems. The proportion of a multi-layered composition of the disclosure comprising the wash layer, either in terms of volume within a reaction vessel or linear depth within a reaction vessel may vary based on the volume and/or depth of the reaction vessel. The wash layer may comprise at least 10%, 20%, 30%, 40%, 50%, 60%, 70%, or 80% of the volume and/or depth of the reaction vessel. One skilled in the art can readily determine, using no more than routine experimentation, the gel depth required to provide sufficient “wash” of magnetic particles comprising bound nucleic acids from the sample being extracted as the particles are drawn from upper extraction mixtures into the wash layer.

Once the gel wash layer has set, an intermediate layer comprising a low-temperature meltable wax is poured on top of the gel wash layer and allowed to set. The low-temperature meltable wax layer forms a seal between the concentrated semi-solid lysis paste and the gel wash layer, thereby preventing moisture from the aqueous gel from contacting the lysis paste and compartmentalizing the lysis reaction from the aqueous component such that the lysis reaction proceeds using moisture derived only from a biological sample. In instances, the low-temperature meltable wax layer may be a single homogenous low-temperature meltable wax layer.

Waxes are hydrocarbons often characterized by the presence of long aliphatic alkyl chains. Natural waxes include plant and animal waxes and derivatives. Synthetic waxes include petroleum derived waxes (e.g. paraffin and microcrystalline wax).

In embodiments, the low-temperature meltable wax layer is comprised of a paraffin wax layer or a derivative thereof. Paraffin wax typically has a melting point in the 50 to 70 degree C. range. The melting point of paraffin wax can be reduced through the addition of mineral oil, for example. As used herein, low-temperature meltable wax refers to wax that is solid at commercial shipping temperatures (e.g., 4-45 degrees C.) but transitions to liquid at the lower end of the paraffin melting point ranges (e.g., 50 to 55 degrees C.). While one skilled in the art could tune melting temperatures of the intermediate layer to melt higher than the preferred range of 50 to 55 degrees C., this would incrementally increase the cost of the assay, in terms of energy input. Additionally, such an increase in temperature is not preferred from the standpoint of integrity of the biological sample and components thereof. These disadvantages notwithstanding, a multi-layered composition with an intermediate layer comprising a meltable wax layer melting above 55 degrees C. falls within the scope of the present disclosure, provided that the layer melts and participates in the density inversion as described herein.

Microcrystalline waxes, commonly used in the custom making of jewelry, also find application in the present disclosure. Microcrystalline wax, as compared to paraffin, contain a higher percentage of isoparaffinic (branched) hydrocarbons and napthenic hydrocarbons. It is generally darker, more viscous, denser, tackier and more elastic than paraffin waxes. The melting point of microcrystalline wax can be reduced through the addition of mineral oil, for example.

As an alternative to the single homogenous layer of low-temperature meltable wax, the intermediate layer may comprise a plurality of low-temperature meltable wax layers, wherein each of the plurality of low-temperature meltable wax layers is separated by an intervening sealing layer which serves one or more functions. The intervening sealing layer or layers aid in the staged release of the plurality of low-temperature meltable wax layers following the application of heat sufficient to melt the low-temperature meltable wax layers. An advantage of staged release of the plurality of low-temperature meltable wax layers is to promote additional mixing of the lysis reaction. Additionally, the intervening sealing layer or layers may provide enhanced hydrophobic sealing thereby further insulating the lysis paste from liquid present in the wash layer.

As will be discussed in greater detail below, following the initial hydration of the lysis paste with the biological sample, heat is applied to the exterior of the reaction vessel in order to melt low-temperature meltable wax layers and intervening sealing layers (in embodiments wherein the intervening sealing layer is a solid or semi-solid such as a lower-temperature meltable wax). Heat may be applied via a heating element to the entire reaction vessel simultaneously or may be applied to only a portion of the reaction vessel. In some embodiments, the heating element is designed to fit in an instrument, including but not limited to an automated analysis instrument. In some embodiments the heating element may be permanently affixed within an instrument, or may be removable. In some embodiments, the heating element is a heat block configured with holes to receive one or more reaction vessels, though other heating element configurations such as coils or air are contemplated. In some embodiments, the holes extend the entire depth of the heating block, such that the reaction vessel protrudes through the bottom of the heating block. In some embodiments, the heat block may be manually manipulated and may be heated by means of a hot plate or laboratory oven. Heating blocks may be made of any material that retains and transfers heat, including but not limited to metal (e.g., aluminum).

In the case of a single low-temperature meltable wax layer, melting tends to take place at the internal surface of the reaction vessel and, once sufficient melting has taken place, the remaining plug of low-temperature meltable wax is released and, through density inversion, the released plug rises through the through the higher-density solubilized concentrated semi-solid lysis paste thereby mixing the lysis reaction comprising the liquid biological sample, the solubilized concentrated semi-solid lysis paste, and the plurality of metal oxide coated magnetic particles. This mixing can eliminate the need for mechanical agitation in the performance of the relevant assay. The mixed lysis reaction sinks beneath the melted wax and rests on top of the gel wash layer, in fluid communication with the gel wash layer.

In instances wherein the intermediate layer comprises a plurality of low-temperature meltable wax layers, the principle is the same but the release of each of the plurality of plugs resulting from the melting and release of each of the plurality of low-temperature meltable wax layers is staged in sequence to facilitate multiple rounds of mixing, and to provide enhanced insulation of lysis paste from aqueous gel during storage. In some embodiments of compositions and methods of the disclosure, each of the plurality of low-temperature meltable wax layers is separated by an intervening sealing layer. As described elsewhere herein, the intervening sealing layers aid in staging sequential melting of low-temperature meltable wax layers by physically separating the low-temperature meltable wax layers. Such physical separation tends to insulate adjacent layers of low-temperature meltable wax within the intermediate layer. Therefore, when heat is applied to the reaction vessel from the top, the topmost low-temperature meltable wax layer will release first and mix the lysis reaction comprising the biological sample, the solubilized concentrated semi-solid lysis paste and the plurality of metal oxide coated magnetic particles as described. The mixed lysis reaction sinks beneath the melted wax to rest on top of the gel wash layer, in fluid communication with the gel wash layer. The intervening sealing layer tends to insulate the next lowest low-temperature meltable wax layer such that it will not release simultaneously with the topmost low-temperature meltable wax layer. One of skill in the art will understand that, using no more than routine experimentation, adjusting the volume and/or thickness of each low-temperature meltable wax layer and/or of each intervening sealing layer may be used to control the speed with which the melting, and therefore the density inversion-driven mixing of the lysis reaction, occurs.

While the sequential melting of layers, as described in the preceding paragraph offers certain advantages, one skilled in the art will recognize that if heat is applied uniformly along the reaction vessel(s), each of the plurality of low-temperature meltable wax layers and intervening sealing layers will tend to melt and release (i.e., invert) almost simultaneously. It will be recognized that the simultaneous release and inversion of multiple layers will provide effective mixing. Example 3 relates to such an embodiment.

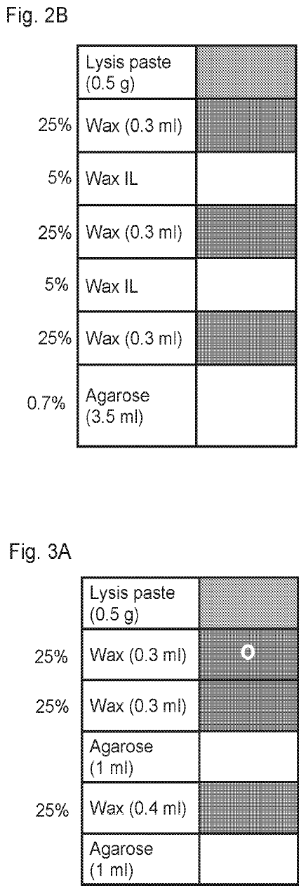

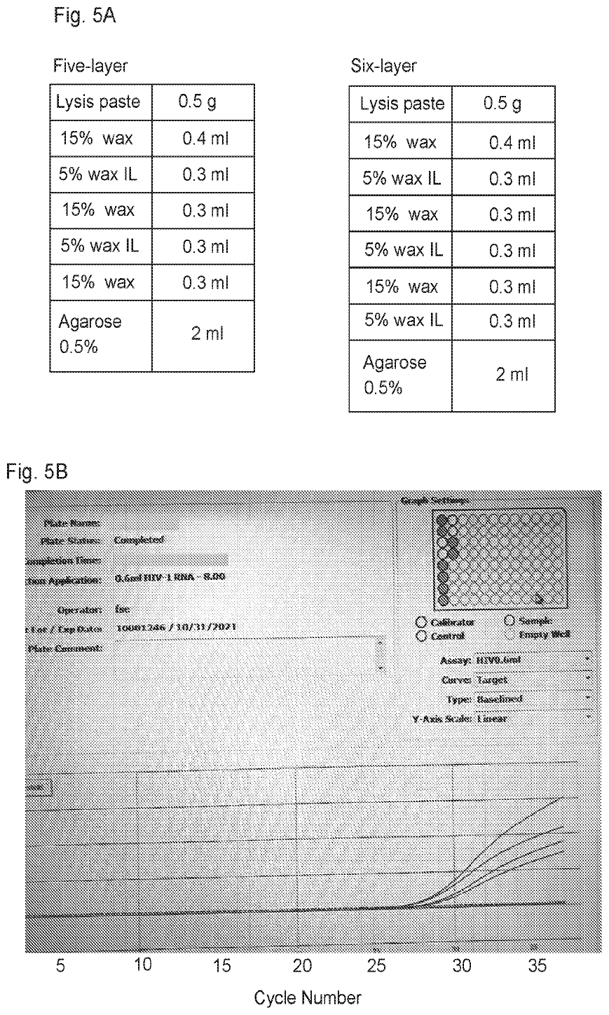

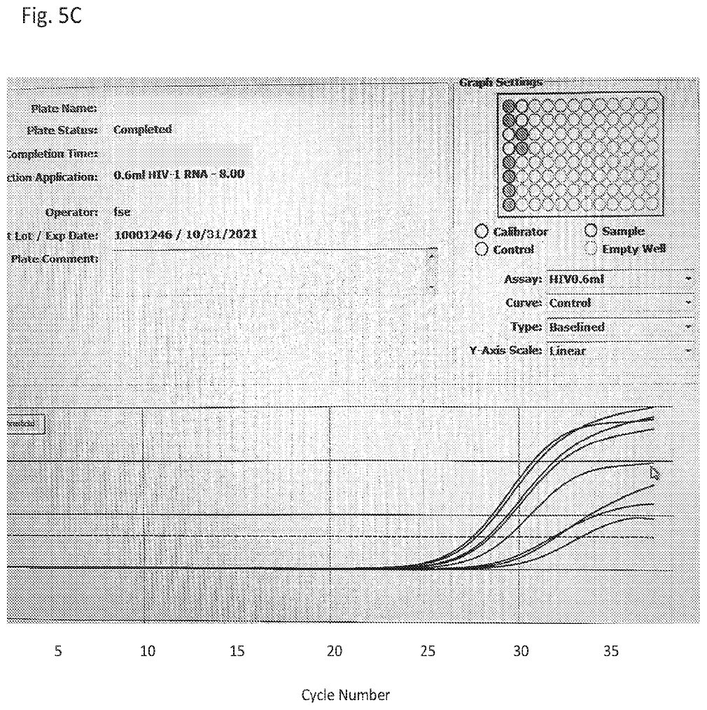

There is no theoretical limit on the number of low-temperature meltable wax layers and corresponding intervening sealing layers that may be used. One of skill in the art will understand, using no more than routine experimentation that the number of low-temperature meltable wax layers and corresponding intervening sealing layers may be constrained by physical factors including but not limited to the volume and/or depth of the reaction vessel, the volume and/or depth of the gel wash layer, the expected biological sample volume to be applied, and a desired overall length of time for the lysis reaction. In a preferred embodiment, at least one of the plurality of low-temperature meltable wax layers is in contact with the gel wash layer and at least one of the plurality of low-temperature meltable wax layers is in contact with the lysis paste layer. In another embodiment, at least one of the one of the plurality of intervening sealing layers is in contact with the gel wash layer and at least one of the plurality of low-temperature meltable wax layers is in contact with the lysis paste layer. In another embodiment, at least one of the one of the plurality of intervening sealing layers is in contact with the gel wash layer and at least one of the plurality of intervening sealing layers is in contact with the lysis paste layer. In some embodiments, agarose gel may be used as an intervening sealing layer, although such a material would not tend to minimize moisture migration from the gel wash layer to the lysis paste layer, as described elsewhere herein. Although low-temperature meltable paraffin wax layers formulated to melt in the 55° C. range demonstrate minimal shrinkage from the side walls of the reaction vessel as indicated in the Examples section which follows, even minimal shrinkage can allow for moisture from the aqueous gel layer to pass between the sidewall of the reaction vessel and the shrinking low-temperature meltable wax layer. This moisture transfer is undesirable because any moisture contact with the semi-solid lysis paste layer could partially solubilize the lysis paste layer prior to application of a biological sample. Partially solubilized lysis paste could potentially interfere with the ability of the multi-layered composition to be used to reliably extract nucleic acids from a sample, potentially obscuring experimental or clinical results obtained from the biological sample. Therefore, using a hydrophobic material as an intervening sealing layer may also provide a solution to the moisture migration problem by further insulating the lysis paste from liquid present in the wash layer. In a preferred embodiment, such intervening hydrophobic sealing layers are specifically selected to be a wax that melts at a temperature lower than the melting temperature of the plurality of low-temperature meltable wax layers, but higher than standard commercial shipping temperatures. In some embodiments, the plurality of low-temperature meltable wax layers comprises two low-temperature meltable wax layers separated by an intervening hydrophobic sealing layer that melts at a lower temperature. In some embodiments, the plurality of low-temperature meltable wax layers comprises three low-temperature meltable wax layers separated by two intervening hydrophobic sealing layers that melt at a lower temperature (as illustrated in FIG. 2A). In some embodiments, the plurality of low-temperature meltable wax layers comprises four low-temperature meltable wax layers separated by three intervening hydrophobic sealing layers that melt at a lower temperature. For example, though not intended to be limiting, in one embodiment (as illustrated in FIG. 2B) the intermediate layer comprises three low-temperature meltable wax layers, each of which melts at 55° C. (0.3 ml, 25% paraffin/75% mineral oil), and two intervening lower-temperature meltable wax layers, each of which melts at 45-50° C. (0.3 ml, 5% paraffin/95% mineral oil). In some embodiments, the plurality of low-temperature meltable wax layers comprises three low-temperature meltable wax layers separated by two intervening hydrophobic sealing layers that melt at a lower temperature, and further separated from the gel wash layer by an intervening hydrophobic sealing layer that melts at a lower temperatures. In other non-limiting examples of embodiments (as illustrated in FIG. 5A), the intermediate layer comprises three low-temperature meltable wax layers, each of which melts at 55° C. (0.3 ml or 0.4 ml, 15% paraffin/85% mineral oil), and two intervening lower-temperature meltable wax layers, each of which melts at 45-50° C. (0.3 ml, 5% paraffin/95% mineral oil); or, the intermediate layer comprises three low-temperature meltable wax layers, each of which melts at 55° ° C. (0.3 ml or 0.4 ml, 15% paraffin/85% mineral oil), and three intervening lower-temperature meltable wax layers, each of which melts at 45-50° C. (0.3 ml, 5% paraffin/95% mineral oil).

One of skill in the art will recognize that a variety of wax blends can be formulated to function as an intervening hydrophobic sealing layer that melts at a lower temperature than a neighboring low-temperature meltable wax layer. For example, Bio-Rad® Chill-out™ liquid wax solidifies when chilled below 10 degrees C., and as described elsewhere herein, may be used to dilute paraffin to produce a paraffin wax blend with a lower melting temperature than that of undiluted paraffin. As described elsewhere herein, paraffin or a microcrystalline wax may be diluted with mineral oil (also known as paraffin oil) lowers the melting temperature of paraffin. Using no more than routine experimentation, one of skill in the art will be able to formulate wax blends for intervening hydrophobic sealing layers that melt at prescribed temperatures. Both of the foregoing alternatives tend to minimize the migration of moisture from the wash layer to the lysis paste layer. Alternatively, in a preferred embodiment, a mineral oil layer may serve as an intervening hydrophobic sealing layer.

Additionally, one of skill in the art will understand that choosing a material or material for intervening sealing layers, particularly for intervening hydrophobic sealing layers, may be at least partially influenced by one or more properties of an intended reaction vessel. Non-limiting properties of an intended reaction vessel include size, shape, chemical composition (such as glass or polymer composition), and thermal conductivity. Using no more than routine experimentation, one of skill in the art will be able to identify hydrophobic materials that form a seal against an intended reaction vessel sufficient to prevent moisture migration under a required set of conditions.

A sealing material used in embodiments of compositions and methods of the disclosure has several important properties. The sealing material preferably provides a substantially impermeable seal, which, as used herein, means that the sealing material preferably does not crack or shrink when in contact with caustic substances (non-limited examples include acids and bases) or at temperatures below the desired melting temperature, including at temperatures below room temperature, even, for example, at 4° C. If solid or semi-solid, the sealing material preferably also has a predictable melting temperature that is compatible with the temperature range for the lysis reaction, for example, beginning to melt at least at temperatures of or above 50-55° C. In addition, the melted sealing material must have a lower density than the semi-solid lysis paste, allowing the melted sealing material to rise and contribute to mixing of the lysis reaction, and allowing the lysate to sink. Furthermore, the sealing material may not bind to or otherwise interfere with the components of the lysis reaction or nucleic acids from the liquid biological sample. One of skill in the art will be able to identify polymers or other types of materials that meet these conditions.

The uppermost layer of the multi-layered composition of the disclosure comprises a concentrated semi-solid lysis paste (also referred to herein as “lysis paste”). In a disclosed embodiment, the lysis paste is comprised of guanidinium thiocyanate (GITC), Tris-HCl, Tris base, and Tween®-20. One skilled in the art will recognize that other reducing and denaturing agents can be used in the lysis paste in addition to, or instead of, GITC. Such alternative reducing and denaturing agents are available commercially from ThermoFisher Scientific. Alternatives to the liquid detergent Tween®-20 may also be used. One skilled in the art will recognize SDS and Triton X-100 as exemplary of substitute liquid detergents. Similarly, alternative buffer systems may be employed in place of Tris-HCL, Tris base. One skilled in the art will recognize that the pH range covered is not the sole consideration with regard to buffer selection.

The concentrated semi-solid lysis paste of the instant disclosure has multiple significant differences in comparison to previously described lysis buffers (U.S. Pat. No. 6,936,414 B2, the disclosure of which is incorporated herein by reference). All conventional lysis buffers are aqueous liquids, meaning that their components have been dissolved in water. As aqueous liquids, conventional lysis buffers are therefore dispensed by pipettes, pumps, or other fluid management systems. However, the concentrated semi-solid lysis paste of the disclosure is not water-based. In contrast to convention lysis buffers, the concentrated semi-solid lysis paste uses powdered solid chemical components mixed with a liquid detergent, yielding a mixture that is a thick slurry and/or a dense foam with very small bubbles. Therefore, the concentrated semi-solid lysis paste is stable, does not flow, does not leak from a reaction vessel, and can be dispensed using common paste extrusion methods well known and used for semi-solid materials such as clay, polymers, and foodstuffs. Non-limiting examples of paste extrusion methods include a cookie press and a wide-bore syringe.

In aspects of the disclosure, GITC, Tris-HCl, and Tris base are provided as solid components and are finely powdered and sifted before being mixed with a detergent. In some embodiments the detergent is Tween®-20 (polyethylene glycol sorbitan monolaurate). In some embodiments, Tween®-20 may be used at levels of at least 10%, at least 15%, at least 16%, or at least 20%, (w/w) to mix together the solid components. In some embodiments, Tween®-20 is added to the solid components at 16%. In some embodiments, the mixture of GITC, Tris-HCl, Tris base, and Tween®-20 is calculated to give 3.8 M GITC, 100 mM Tris (pH 7.8), and 8% (w/w) Tween®-20 when mixed 1:1 w/v with a sample. In some embodiments, the concentrated semi-solid lysis paste has a residual moisture of less than 10% and does not flow. In certain embodiments, the concentrated semi-solid lysis paste does not flow, and has a residual moisture of approximately 5%, approximately 4%, approximately 3%, approximately 2%, or approximately 1%. In some embodiments, the lysis paste does not separate when heated and is stable at room temperature for at least one year.

In aspects of the disclosure, the lysis paste comprises a plurality of magnetic particles comprising metal oxides that bind nucleic acids during extraction and purification of nucleic acids from a biological sample. Metal oxides bind nucleic acids by binding nucleic acid phosphate groups to the metal oxide.

U.S. Pat. No. 6,936,414 (the “414 patent”), the disclosure of which is incorporated herein by reference, discloses the reversible binding of nucleic acids to a metal oxide support material. Disclosed support materials included “particles”. The use of metal oxide support particles, as taught in the '414 patent, provides several important advantages conventional sample preparation methods. For example, metal oxides have a high affinity for nucleic acid sequences and therefore sample-to-sample contamination is minimized because nucleic acid can controllably be bound to the metal oxide particles without escaping to undesired areas. Additionally, metal oxide supports provide for a more quantitative purification of nucleic acid in a test sample and therefore even small amounts of a desired nucleic acid that may be present in the test sample are collected. Moreover, metal oxide particles can be employed to separate nucleic acid from a test sample with low organic-solvent concentrations (or, significantly, without the use of organic solvents) such as alcohol, phenol or chloroform, which are commonly employed according to other sample prep methods, but pose significant disposal concerns.

Further, nucleic acid can be eluted from metal oxide particles using buffers that are completely compatible with amplification reactions. In other words, nucleic acid separated from a test sample in the manner provided herein directly can be employed in an amplification reaction without the need to exchange the elution buffer with a buffer compatible with an amplification reaction.

Additionally, the metal oxide particles disclosed can be employed to separate both DNA and the various forms of RNA from a single test sample. Hence, the method which utilizes metal oxide particles can be employed to separate nucleic acid from various different cells and/or organisms in the same test sample such that it later can be detected.

Generally, the present disclosure describes contacting a test sample (i.e., a biological sample) with a plurality of metal oxide particles in an extraction buffer. In the presence of extraction buffer, nucleic acid of all types, such as DNA and the various forms of RNA, contained in the test sample bind the metal oxide particles. The metal oxide particles, and any nucleic acid bound thereto, then can be separated from the test sample.

As used herein, the term “metal oxide” refers to oxides and hydroxides of metallic elements in any of their various valence states. Thus, for example, oxides of aluminum, magnesium, titanium, zirconium, iron, silicon, nickel, chromium, zinc and combinations of the forgoing are metal oxides. In some embodiments, the metal or metal oxide is AlTi, CaTi, CoTi, Fe₂Ti, Fe₃Ti, MgTi, MnTi, NiTi, SnTi, ZnTi, Fe₂O₃, Fe₃O₄, Mg, Mn, Sn, Ti, or Zn (e.g., anhydride or hydrated forms) as described elsewhere (U.S. Pat. No. 10,526,596, the disclosure of which is incorporated herein by reference).

In some embodiments, the magnetic particles are copper-titanium oxide-coated (CuTi) magnetic particles, as described elsewhere (U.S. Pat. No. 10,392,613, the disclosure of which is incorporated herein by reference). The present disclosure in not limited to particular amounts of copper and titanium. In some embodiments, the CuTi is present at a ratio of approximately 2:1 Cu to Ti (e.g., 3:1, 2:1, 1:1, 1:2, 1:3, etc.). In some embodiments, the particles have a diameter of 0.5 to 50 μm (e.g., 0.5 μm, 1.0 μm, 1.5 μm, 2.0 μm, 5.0 μm, 10.0 μm, 20.0 μm, 30.0 μm, 40.0 μm, 50.0 μm, etc.). In some embodiments, particles and/or solid surfaces are comprised of organic polymers such as polystyrene and derivatives thereof, polyacrylates and polymethacrylates, and derivatives thereof or polyurethanes, nylon, polyethylene, polypropylene, polybutylene, and copolymers of these materials. In some embodiments, particles are polysaccharides, in particular hydrogels such as agarose, cellulose, dextran, Sephadex, Sephacryl, chitosan, inorganic materials such as e.g. glass or further metal oxides and metalloid oxides (in particular oxides of formula MeO, wherein Me is selected from, e.g., Al, Ti, Zr, Si, B, in particular Al₂O₃, TiO₂, silica and boron oxide) or metal surfaces, e.g. gold. In some embodiments, particles are magnetic (e.g., para-magnetic, ferrimagnetic, ferromagnetic or superparamagnetic). In some embodiments, the particles may have a planar, acicular, cuboidal, tubular, fibrous, columnar or amorphous shape, although other geometries are specifically contemplated. In some embodiments, commercially available particles (e.g., obtained from ISK Magnetics, Valparaiso, IN.; Qiagen, Venlo, The Netherlands; Promega Corporation, Madison, WI; Life Technologies, Carlsbad, CA; Ademtech, New York, NY; and Sperotech, Lake Forest, IL).

In aspects of the disclosure, binding nucleic acids comprises the step of contacting the biological sample being lysed within the lysis paste with a plurality of metal oxide coated magnetic particles. In some embodiments, the plurality of metal oxide coated magnetic particles is added to the semi-solid lysis paste with a minimum of fluid. In aspects of compositions and methods of the disclosure, the order in which the plurality of metal oxide particles is added to the lysis paste relative to the layering of the biological sample may depend on multiple factors, including but not limited to the composition of the sample to be lysed, and whether the experiments to be performed will be performed on a benchtop or in an automated analysis instrument. In some embodiments, the plurality of metal oxide particles is added to the lysis paste as the paste is being prepared, prior to the addition of the paste to a reaction vessel as the uppermost layer. In some embodiments, the plurality of metal oxide particles is added to the lysis paste separately, either after the paste has been added to the reaction vessel but prior to layering of a biological sample above the paste, or after layering of the biological sample above the paste. In some embodiments, the plurality of metal oxide particles is added to the biological sample prior to layering the biological sample above the lysis paste. In preferred automated embodiments, the plurality of metal oxide particles are on-board an automated analysis instrument in bulk and are added automatically.

In some embodiments, the plurality of metal oxide particles are present in a quantity calculated to represent a molar excess relative to the quantity of nucleic acids calculated to be present in a biological sample. In some embodiments, the plurality of metal oxide particles may be added to the lysis paste with minimal fluid to give 1 mg particles per 0.5 g lysis paste. In some embodiments, metal oxide particles are added at suspensions of 15% particles. In some embodiments, the plurality of metal oxide particles are titanium oxide particles, and in some embodiments, the titanium oxide particles are copper titanium (CuTi) coated particles.

When a biological sample is added to the lysis paste, the lysis reaction begins as moisture in the sample dissolves the paste, releasing the small bubbles within the paste whose movement begins to mix the paste, sample, and magnetic particles. As described elsewhere herein, a biological sample may be a naturally occurring liquid biological sample, non-limiting examples of which include blood, plasma, and saliva, or may otherwise be prepared for use such that it comprises moisture, non-limiting examples of which include a suspension of cells or homogenized tissue in a buffer, and cell culture supernatant. As described elsewhere herein, the rising of the lower-density meltable wax layers as they melt further contributes to mixing the paste, sample, and magnetic particles, thereby obviating need for mechanical agitation. As sample lysis proceeds, the detergent breaks down cell membranes, releasing nucleic acids into the reaction, where they are contacted and bound by the metal oxide magnetic particles in the presence of the chaotropic reagent GITC. Chaotropic agents are well known in the art and include entities that break down, or solubilize, proteins. Exemplary chaotropic reagents include, but are not limited to guanidine isothiocyanate (GITC), guanidine HCl, potassium iodide, urea, and the like. Once all layers of the intermediate layer have melted and risen through and above the lysis paste, the lysis reaction will have sunk through the reaction vessel such that it then rests upon and contacts the wash layer. In some embodiments, heat will be applied to the reaction vessel for at least 1, 2, 3, 4, 5, 10, or 15 additional minutes to ensure that the lysis reaction proceeds to completion. Once the lysis reaction is complete, a magnetic force is applied as described elsewhere herein to draw the metal oxide magnetic particles with bound nucleic acids through the aqueous gel of the wash layer to remove contaminants from the lysis reaction.

The terms “nucleic acid” or “nucleic acids” as used herein refer to a polymer comprising multiple nucleotide monomers. The term “nucleotide” as used herein includes a phosphoric ester of nucleoside—the basic structural unit of nucleic acids (DNA or RNA). A nucleic acid may be either single stranded, or double stranded with each strand having a 5′ end and a 3′ end. A nucleic acid may be RNA, DNA (including but not limited to cDNA or genomic DNA), or hybrid polymers (e.g., DNA/RNA). The terms “nucleic acid” and “nucleic acids” do not refer to any particular length of polymer. Nucleic acids used in embodiments of compositions and methods of the disclosure may be at least 1, 2, 3, 4, 5, 10, 20, 30, 40, 50, 60, 70, 80, 90, 100, 200, 500, 1000, or 2000 kb, or more in length. The term “sequence,” used herein in reference to a nucleic acid, refers to a contiguous series of nucleotides that are joined by covalent bonds, such as phosphodiester bonds. A nucleic acid may be chemically or biochemically synthesized, or may be isolated from a subject, cell, tissue, or other biological sample or source that comprises, or is believed to comprise, nucleic acid sequences including, but not limited to RNA, mRNA, and DNA. Nucleic acids enriched, isolated, or purified using the composition of the present disclosure may be used in any conventional molecular assay or process known to those of ordinary skill in the art because the nucleic acids are not altered in any way that may be detrimental to their subsequent use. For example, the nucleic acids may be sequenced, amplified by PCR, used in expression vectors, etc. In this regard, the nucleic acids may be contacted with enzymes such as, for example, a DNA polymerase or a reverse transcriptase after passing through the wash layer. Further, this disclosure contemplates that the nucleic acid is sequenced on the solid substrate without dilution. Further, this disclosure contemplates that the nucleic acids bound to the solid substrate are contacted with bisulfite after passing through the wash layer such that unmethylated cytosines are deaminated. Further, this disclosure contemplates that at least one nucleic base in the nucleic acid has an epigenetic modification.

In some embodiments, the intermediate layer comprises an internal control. An internal control is a known nucleic acid sequence with which the performance of a subsequent assay, such as reverse transcription PCR (RT-PCR), real-time RT-PCR (rRT-PCR), or quantitative RT-PCR (qRT-PCR), has been tested and confirmed. One of skill in the art will know how to choose an appropriate internal control based on the types of samples to be used, the assay or assays to be performed, the type or types of nucleic acids to be assayed, and the nucleic acid sequences to be determined.

Conventional silica preparation compositions and methods for binding and isolating nucleic acids rely on salting out nucleic acids onto silica surfaces, and require washes with high levels of ethanol or other alcohols to remove lysis buffer contaminants. Those alcohols must also be removed by drying before elution due to the inhibitory nature of the alcohols in PCR-based tests. In contrast to conventional silica compositions and methods, metal oxide coated magnetic particles, including CuTi particles, retain nucleic acids under very low ionic strength conditions, allowing them to be washed with water to remove contaminants without eluting bound nucleic acids. Furthermore, metal oxide particles, including CuTi particles, do not require alcohol for sample processing washes and do not require any drying steps prior to elution. Consequently, those properties allow metal oxide particles, including CuTi particles, to be washed to remove lysis contaminants by being magnetically drawn through a low ionic strength aqueous gel as described elsewhere (U.S. Pat. No. 9,803,230, the disclosure of which is incorporated by reference herein). Conventional silica methods use water or low ionic strength buffers to elute nucleic acids and therefore cannot use the low ionic strength aqueous gel to remove contaminants. Metal oxide particles, including CuTi particles, use a low ionic strength phosphate buffer (for example, a 5 mM phosphate buffer) to elute nucleic acids; such a buffer is not inhibitory to subsequent reactions in which the eluted nucleic acids may be used, including but not limited to PCR.

An “elution buffer” according to the present disclosure can be any reagent or set of reagents that separates bound nucleic acid from the metal oxide of the CuTi particle or other metal oxide magnetic particle. In some aspects, compositions and methods of the disclosure comprise an elution buffer as a layer adjacent to and in fluid communication with the gel wash layer. In some embodiments, the elution buffer is a low ionic strength elution buffer that uses a phosphate counter-ion to elute the nucleic acids. In some embodiments, the low ionic strength elution buffer is a phosphate buffer, for example, a 5 mM phosphate buffer. In some embodiments, the low ionic strength elution buffer comprises an organophosphate such as phosphoserine. In a preferred embodiment, the low ionic strength elution buffer is an inorganic phosphate.

In some embodiments of compositions and methods of the disclosure, the elution buffer may be beneath the aqueous gel wash layer in the reaction vessel. In some embodiments, the elution buffer may be separated from the aqueous gel wash layer by a low-temperature meltable wax layer. In certain embodiments, the elution buffer may be adjacent to the aqueous gel wash layer. In some embodiments, the elution buffer may be contained in a separate chamber or tube into which the reaction vessel may be inserted, such that metal oxide particles, including CuTi particles, with bound nucleic acids could be magnetically drawn through the aqueous gel wash layer directly into the elution buffer within the separate chamber or tube. In certain embodiments, the bottom of the reaction vessel may be reversibly sealed with a meltable plug (for example, though not limited to, a meltable wax plug) that may be melted at the elution stage allowing the metal oxide particles to be magnetically drawn into an elution chamber. The reaction vessel could then be removed from the elution chamber and the eluate comprising the eluted nucleic acids could then be transferred to an assay or stored for later use. In some embodiments, metal oxide particles with bound nucleic acids may be transferred directly into an assay.

Once a multi-layered composition of the present disclosure is completed, it is ready for immediate use. Alternatively, the upper end of the reaction vessel can be sealed and the multi-layered composition can be stored for later use. An important consideration in the design of the multi-layered composition is shelf-life. The multi-layered composition should be stable for at least 6 months, and preferably for multiple years. Standard commercial storage and shipping temperatures are above the freezing point of gel wash layers described herein and below the melting point of low-temperature melting wax compositions described herein. For example, though not intended to be limiting, commercial shipments are preferably packaged such that the maximum temperature during shipping does not exceed 45° C. and the minimum temperature during shipping does not fall below 4° C. To preserve the integrity of the wash layer, aqueous buffers, such as elution buffers may be provided separately and may not be sealed within the reaction vessel at the time of manufacture.

In aspects, the present disclosure provides methods for extracting and purifying nucleic acids from a biological sample using a multi-layered composition as described elsewhere herein. Multi-layered compositions of the methods comprise a reaction vessel in which the layers are assembled; an uppermost layer within the reaction vessel comprising a concentrated semi-solid lysis paste and a plurality of metal oxide coated magnetic particles, a non-limiting example of which includes CuTi particles; an intermediate layer comprising a low temperature-meltable wax; and a lower wash layer comprising an aqueous gel.

In embodiments, the multi-layered composition of the methods is assembled in a reaction vessel as described above herein, beginning with the lower gel wash layer, followed by the intermediate layer, and then the uppermost layer comprising the lysis paste. As described elsewhere herein, the lysis paste is further provided with a plurality of metal oxide magnetic particles. In some embodiments, the plurality of metal oxide particles is added to the lysis paste as the paste is being prepared, prior to the layering of a biological sample above the lysis paste, or after the layering of the biological sample. In some embodiments, the plurality of metal oxide particles is added to the biological sample. In some embodiments, the bottom of the reaction vessel is reversibly sealed prior to the addition of the lower gel wash layer, for example with a low temperature meltable wax. The multi-layered composition may be assembled manually, for example by pipetting or pouring each layer into the reaction vessel. Alternatively, assembly of the multi-layered composition may be automated, for example, though not intended to be limiting, using a liquid handling robot or a paste extruder. Automated assembly of the multi-layered composition of the methods may be advantageous for production of disposable cartridges for use in automated analysis instruments for rapid and/or large-scale extraction, isolation, and analysis of nucleic acids from biological samples.

In embodiments described elsewhere herein, the reaction vessel has a substantially circular cross-section, and may vary in size depending on whether it is intended for benchtop use or for use with an automated analysis instrument. A circular cross-section is not a requirement as to all embodiments. In some embodiments, the reaction vessel may be a vertically-aligned tube. In some embodiments, the portion of the reaction vessel into which the gel wash layer is formed comprises a bend such that the overall shape of the reaction vessel resembles a “J” or a “U.” Such shapes may be advantageous in the context of automated analysis instruments because they may enable a faster and more efficient workflow, based on available space within a particular automated instrument, as well as how an automated instrument may be able to physically execute the steps of the methods within a particular space and subsequently process metal oxide magnetic particles drawn through the gel wash layer. For example, though not intended to be limiting, a “U”-shaped reaction vessel for use with an automated analysis instrument may be contemplated wherein the first arm of the “U” contains the intermediate layer and lysis paste, and the bend and second arm of the “U” contain the gel wash layer which is further in fluid communication with an elution buffer layered above the gel wash layer. In such a configuration, a biological sample is layered above the lysis paste layer, the reaction vessel is heated and inverted density mixing occurs, and the metal oxide magnetic particles and bound nucleic acids are drawn through the gel wash layer in the bend, which may include being drawn horizontally or otherwise non-vertically, up the second arm of the “U”, and into the elution buffer. In this configuration, the elution buffer is easily accessible to machinery within the automated analysis instrument that transfers the eluted nucleic acids to a subsequent molecular assay. In some embodiments, metal oxide particles may be magnetically drawn along different pathways through an extended gel wash layer and may be drawn into different chambers or cartridges for subsequent nucleic acid amplification and/or detection.

In embodiments, as described above herein, a biological sample is layered above the uppermost layer of lysis paste. As described elsewhere herein, the biological sample may be may be naturally occurring, may be a concentrate or suspension of cells or tissues or fragments thereof in a buffer, may be a products of cells or tissues, may be synthetic nucleic acids, or may be a liquid biological sample. Layering of the biological sample initiates the lysis reaction because moisture from the biological sample solubilizes the lysis paste, converting it from a thick slurry or foam to a liquid, and releasing bubbles trapped within the paste that aid in mixing the solubilized paste and the biological sample. The solubilized lysis paste that further comprises the biological sample and metal oxide particles is also referred to herein as the lysis reaction. As described elsewhere herein, detergent within the lysis reaction breaks down cell and nuclear membranes and any other tissue components within the biological sample, releasing nucleic acids. Nucleic acids released from the biological sample are then available to contact and bind the plurality of metal oxide particles within the lysis reaction.

As described elsewhere herein, the intermediate layer comprises a low-temperature meltable wax and separates the lower aqueous gel wash layer from the uppermost lysis paste layer. In some embodiments the intermediate layer may comprise a single layer of low-temperature meltable wax. In some embodiments, the intermediate layer comprises a plurality of low-temperature meltable wax layers wherein each of the plurality of low-temperature meltable wax layers is separated by an intervening sealing layer that melts at a lower temperature than the layer immediately below it. In some embodiments, the intervening sealing layer is a lower-temperature meltable wax or mineral oil. One having skill in the art will be able to determine using only routine experimentation the optimal number, arrangement, and composition of low-temperature meltable wax layers and intervening sealing layers for a particular use.

In embodiments of methods of the disclosure, heat is applied to the exterior of the reaction vessel to initiate melting of the intermediate layer, resulting in density inversion characterized by the intermediate layer melting and rising through the lysis reaction, thereby mixing the lysis reaction. As described above herein, multiple types of heating elements may be used, including but not limited to heating blocks, coils, or heated air. Heating the reaction vessel may involve but does not require direct contact between the heating element and the reaction vessel. Heat is applied such that the intermediate layer melts sequentially from uppermost to lowermost—that is, such that the portion of the intermediate layer initially in contact with the lysis reaction melts and rises first, and then each subsequent intervening layer or low-temperature melting wax layer melts and rises sequentially. One of skill in the art will understand that heat will need to be applied at a temperature sufficient to melt the intermediate layer, but not high enough to damage the biological sample or interfere with the lysis reaction. One of skill in the art will be able to determine an appropriate temperature and duration with no more than routine experimentation. The mixing phenomenon resulting from density inversion as melted wax of lower density than the lysis reaction rises is advantageous for both benchtop and automated applications because it renders mechanical mixing steps unnecessary, thereby reducing overall workflow time. Density inversion is completed when all layers comprising the intermediate layer, all low temperature meltable wax layers and all intervening sealing layers, have melted and risen through the lysis reaction to the top of the reaction vessel, thereby sealing the top of the reaction vessel. As a result of completed density inversion, the lysis reaction rests on and is in fluid communication with the gel wash layer. As described elsewhere herein, heat may be applied to the reaction vessel for an additional period of time following completed density inversion in order to ensure the lysis reaction is driven to completion.

Once the lysis reaction rests on the gel wash layer, the plurality of metal oxide particles and bound nucleic acids may be attracted by positioning a magnet around or adjacent to the reaction vessel. The application of magnetic force draws the metal oxide particles to the wall of the reaction vessel. A variety of magnet shapes may be used, including but not limited to a bar magnet, donut magnet, or electromagnet. The magnet may be handheld if the method is being manually performed on a benchtop, or may be a programmable component of an automated instrument. One of skill in the art will be able to select a magnet best suited for the intended application. Moving the magnet drives the migration of attracted metal oxide particles and bound nucleic acids at least partially through the wash layer thereby removing extraction contaminants. In some embodiments, further application of the magnetic force may be used to drive the migration of metal oxide particles and bound nucleic acids into an elution buffer after the metal oxide particles and bound nucleic acids have been drawn through the gel wash layer. As described above herein, the elution buffer may be in fluid communication with the gel wash layer, or may be within a separate container. The elution buffer may be a low ionic strength buffer and may further be a phosphate buffer. Some or all of the eluted nucleic acids may be manually transferred to an assay or may be robotically transferred as part of an automated assay performed by an automated molecular analysis instrument. As discussed above herein, the isolated nucleic acids may be used for subsequent molecular assays, including but not limited to PCR, qPCR, and RT-PCR. In some embodiments, some or all of the eluted nucleic acids may be stored for use in future applications. In some embodiments, direct amplification or sequencing of nucleic acids bound to the metal oxide particles is contemplated, wherein after metal oxide particles and bound nucleic acids have been drawn through the gel wash layer, further application of the magnetic force may be used to draw the particles and bound nucleic acids directly into a molecular assay.

### EXAMPLES

### Overview

Described in Examples 1, 2, 3 and 4 below is the development and testing of compositions and methods for the extraction of nucleic acids from a liquid biological sample. A novel nucleic acid extraction system was developed, offering simple operation requiring a minimal number of steps and eliminating many of the complex operations currently needed to extract nucleic acids. Together, the elements create a sophisticated and effective system that combines purification steps in such a way that they are performed simultaneously or in a fashion that reduces or eliminates mechanical steps.

In summary, a low moisture content lysis paste is described in Example 1. In Examples 2 3 and 4, the assembly and use of a layered composition is described. The composition was assembled in polypropylene and polystyrene tubes. Tubes of this type are sometimes referred to herein as a reaction vessel. The uppermost layer in the layered composition is the lysis paste described in Example 1. In use, a liquid biological sample was added from the top of the layered composition, thereby solubilizing the lysis paste. Because the layered composition is intended have an extended shelf life following assembly, and because at least one other layer in the layered composition is an aqueous layer (the aqueous gel wash layer described below in Example 2), it is important that there is no aqueous communication (i.e., leaching) between the aqueous gel layer and the lysis paste layer prior to use of the layered composition for the detection of nucleic acids.

Described below in Examples 2 and 3 is the assembly and use of one or more layers of a low-temperature meltable wax, immediately below the lysis paste layer in the layered composition. This low-temperature meltable wax layer serves as a barrier between the aqueous gel layer and the lysis paste layer thereby preventing aqueous communication (i.e. leaching) between the aqueous gel layer and the lysis paste layer.

In use, the liquid biological sample is added to the layered composition contained within the tubes described in Examples 2 and 3. The liquid biological sample solubilizes the lysis paste which results in the lysis of cells contained with the biological sample. The lysis paste also contains particles coated with a metal oxide, in this case, the metal oxide comprises copper and titanium. Coated particles comprising a copper and titanium metal oxide are referred to therein as CuTi particles. Nucleic acids present in the solubilized lysis paste, primarily nucleic acids released from lysed cells within the biological sample, bind to the CuTi particles thereby forming a CuTi particle/nucleic acid complex that is subject to the attractive force of a magnetic field.

As noted above, the one of more low-temperature meltable wax layers prevent aqueous communication between the aqueous gel wash layer and the lysis paste layer, thereby maintaining the low moisture content property of the lysis paste during storage prior to use. In use, the low-temperature melting property of the one or more wax layers is exploited for the purpose of mixing the solubilized lysis paste which was solubilized by the addition of the liquid biological sample. More specifically, the layered composition is heated to a temperature resulting in melting of the low-temperature meltable wax. Once freed from the walls of the tube within which the layered composition is assembled, through melting, a density inversion takes place and the low-temperature meltable wax layer rises up through the solubilized lysis paste, thereby mixing the constituents thereof.

If the layered composition comprises only a single low-temperature meltable wax layer, the melting and inversion of that layer also establishes aqueous communication between the solubilized lysis paste containing the CuTi particle/nucleic acid complexes, and the aqueous gel layer. A magnetic field is introduced by placing a magnet adjacent the reaction vessel (i.e., the polypropylene or polystyrene tubes of Examples 2 and 3). The CuTi particle/nucleic acid complexes are then drawn from the solubilized lysis paste into the aqueous gel layer thereby removing lysis paste components from the CuTi particle/nucleic acid complexes. From the aqueous gel layer, the CuTi particle/nucleic acid complexes can be drawn into an elution buffer for release of the nucleic acids from the complexes for further analysis.

### Example 1. Preparation of Concentrated Semi-Solid Lysis Paste

A concentrated semi-solid lysis paste (hereafter “lysis paste”) reagent with high levels of a chaotropic salt and detergent, as well as minimal moisture, was developed for nucleic acid extraction.

### Materials

Rather than a liquid lysis reagent, dry guanidinium thiocyanate (GITC), Tris-HCl, and Tris base (Sigma-Aldrich, St. Louis, MO) were powdered and sifted through a 200-micron filter. Tween®-20 (Polyethylene glycol sorbitan monolaurate, CAS 9005-64-5, Sigma-Aldrich, St. Louis, MO) was added at 16% w/w to the sifted dry reagents to create a thick slurry. The mixture of reagents was calculated to give 3.8 M GITC, 100 mM Tris (pH 7.8), and 8% (w/w) Tween®-20 when mixed 1:1 w/v with a sample. CuTi particles (Abbott Molecular, Abbott Park, Ill.) were added with minimal fluid to give 1 mg particles per 0.5 g lysis paste.

### Results

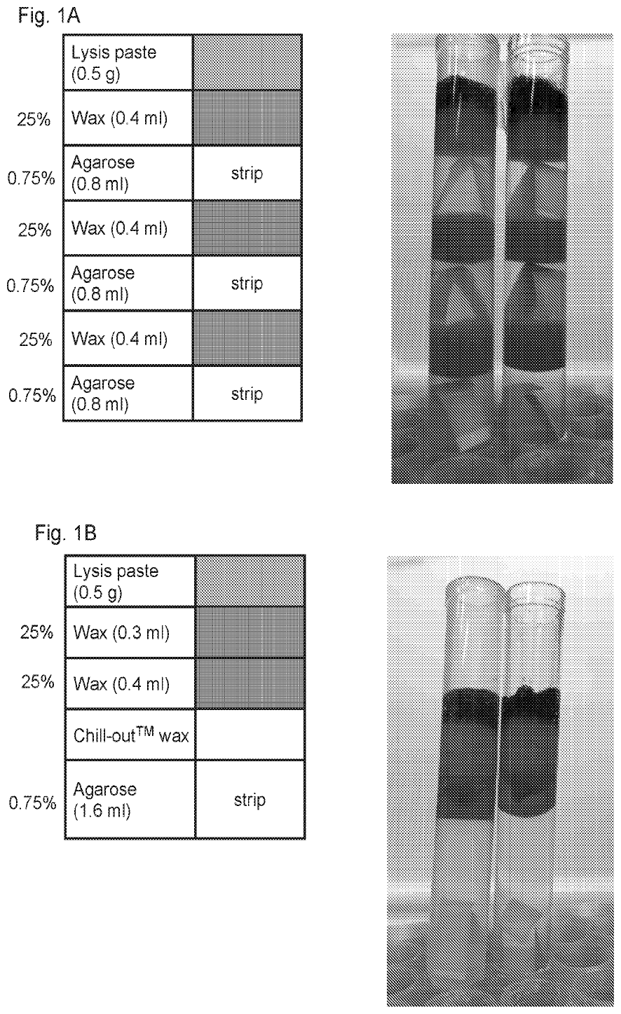

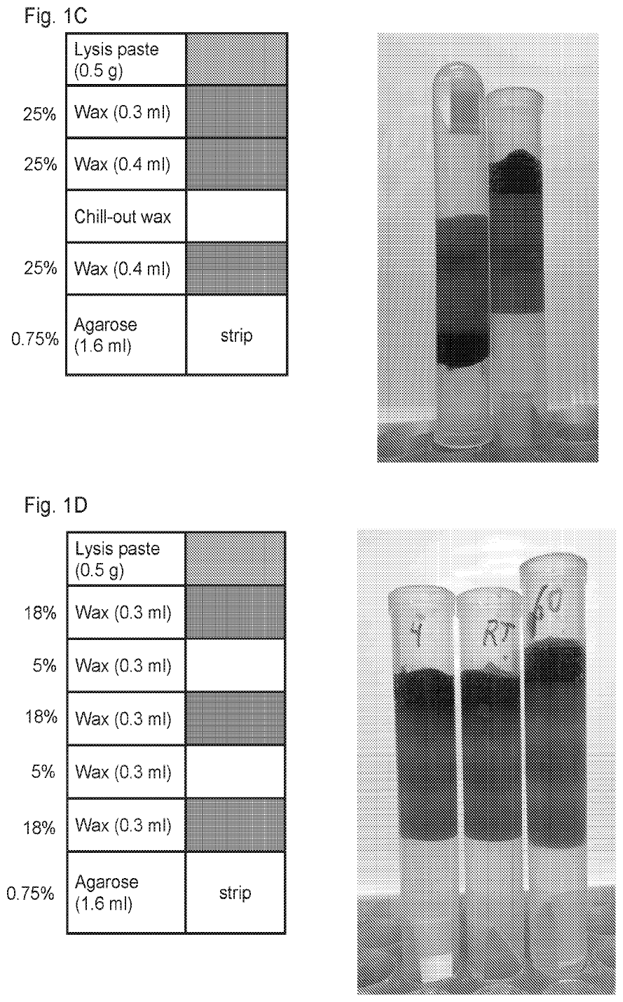

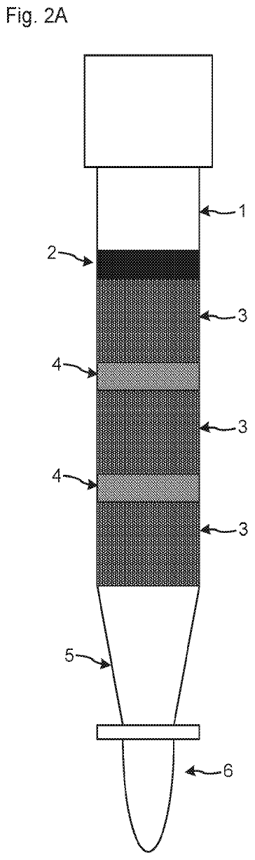

A thick slurry was created by adding Tween®-20 to the sifted dry reagents. The slurry was a foam with very small bubbles that aid in solubilization of the lysis paste by a liquid sample. The lysis paste contained <5% residual moisture and did not separate when heated. Lysis paste was stable at room temperature for more than 12 months and for at least 12 hours at 53ºC. The lysis paste did not flow, and was retained in lysis wells and other containment vessels even after inverted impacts and extended periods of inversion (as shown in FIG. 1C, right panel).

### Example 2. Testing and Experimental Verification of Nucleic Acid Extraction System Components and Methods

### Materials

### Concentrated Semi-Solid Lysis Paste

Lysis paste reagent containing CuTi particles was prepared as described in Example 1.

### Wax Compositions

Wax compositions were prepared and tested as described below herein.

### Aqueous Gel

1% agarose gel was prepared by mixing 100 ml water and 1 g agarose and heating the mixture on a hot plate until boiling and until the agarose was thoroughly dissolved.

### Data Analysis

Data analysis was performed with proprietary Abbott software and with JMP software (SAS Institute, Cary, NC).

### Experiments and Results

### Testing Hydrophobic Wax Compositions

To prevent the minimal moisture lysis paste from contacting the aqueous gel used for “washing” magnetic particles, hydrophobic wax compositions were tested as a material capable of forming a seal between the lysis paste and the aqueous gel layer that did not shrink after cooling, was impermeable at room temperature and at standard shipping temperatures, and melted at a known temperature. Multiple wax compositions were tested to determine their ability to maintain those properties.

### Testing Paraffin/Chill-Out™ Wax Solutions

A 1:1 (w/w) mix of paraffin wax (melting point >65° C.; Sigma-Aldrich, St. Louis, MO) and Chill-out™ Liquid Wax (BioRad, Hercules, CA) was prepared by heating 50 g of each until the paraffin melted and was thoroughly mixed with the Chill-out™ wax. To test the integrity of the wax composition and seal, experimental tubes were prepared with water, acetic acid, and sodium hydroxide.

The indicator regions of pH strips were cut out, placed into the bottom of RV tubes, and covered with 2.5 ml of 1% agarose. 300 μl of the 1:1 wax mixture was layered on top of the cooled agarose and cooled. Either 1 ml of water, 1 M acetic acid, or 1 M NaOH was added to each tube above the wax and the tubes were left overnight. Wax in the 1M acetic acid tube appeared to have separated from the walls of the tube, but the wax in the water and 1M NaOH tubes appeared to be intact.

A 25% (w/w) mixture of paraffin and Chill-out™ wax was also prepared to determine whether the softer wax mixture would also form an intact seal against tube walls. The 25% wax was prepared by melting 24 g of the 50% wax with 24 g of Chill-out™ wax. 5 ml pipette tips were used as extraction tubes. 0.4% agarose was prepared in System Diluent, 50 ml with 0.2 g agarose, and let cool but not harden. Melted 25% wax was held at ˜75° C., and the bottom of the pipette tip was sealed by dipping in wax and letting the wax harden. 75 μl elution buffer was added, and a small amount of wax was added above the elution buffer to seal it. Next, 0.75 ml of agarose was added and allowed to cool and solidify. A 0.2 ml layer of 25% wax was added and allowed to cool, 17 μl HIV internal control was added, and an additional 0.2 ml layer of 25% wax was added and allowed to solidify. 0.5 g lysis paste was added to the top and the tubes stored in an upright position.

### Continued Integrity Testing

Layered tubes were prepared for wax integrity tests (FIGS. 1A-C). As shown in FIG. 1A, both polypropylene and polystyrene tubes were prepared by alternating three layers of 0.75% agarose containing an embedded pH strip (ColorpHast pH 5.0-10.0, EMD MilliporeSigma, Burlington, MA) with three layers of 25% wax (25% paraffin/75% Chill-out™ wax). 0.5 g lysis paste was added on top of the final wax layer. Tubes were left at room temperature for at least one year and periodically examined for shrinkage and seal integrity. If shrinkage or other loss of seal integrity occurred such that lysis paste was able to diffuse and come into contact with one or more agarose layers, a color change in one or more pH strips would be expected. No shrinkage or loss of seal integrity was observed, either through visual examination of the wax layers or by a change in the color of the pH strips.

Additional tubes were prepared with different wax layer configurations (FIGS. 1B-C), again in both polypropylene and polystyrene tubes, and again with pH strips embedded in 0.75% agarose. As shown in FIG. 1B, a diffusion test was prepared with a layer of liquid Chill-out™ (BioRad) wax between the agarose and 25% wax layers, and as shown in FIG. 1C, a layer of liquid Chill-out™ wax was placed between 25% wax layers. Tubes were left at room temperature for at least one year and periodically examined for shrinkage and seal integrity. No shrinkage or loss of seal integrity was observed, and the layer of pure Chill-out™ wax did not make a noticeable difference to seal integrity.

### Testing Paraffin Wax-Mineral Oil and Microcrystalline Wax-Mineral Oil Compositions

Paraffin was observed to shrink after cooling, which could cause a defective seal. Paraffin-mineral oil mixes were therefore tested. Two types of paraffin wax were tested, one that melted at 58-62° C. (Paraffin Wax MP 58-62C, Sigma-Aldrich, St. Louis, MO) and one that melted at >65° C. (Paraffin Wax MP>65 C, Sigma-Aldrich, St. Louis, MO). Each paraffin wax was tested mixed with either light mineral oil (Mineral Oil Light, Sigma-Aldrich, St. Louis, MO) or heavy mineral oil (Mineral Oil Heavy, Avantor Performance Materials, Radnor, PA).

To compare performance, 50 grams of wax-oil combinations were made at different proportions of wax and oil (Table 1). Wax-mineral oil combinations were melted in the laboratory oven at greater than 80° C. Approximately 3 ml of wax mixtures were added to 5 ml reaction vessels and allowed to cool at room temperature. Reaction vessels were placed in a Thermomixer (without mixing) and the temperature was gradually raised by adjusting the setting on the Thermomixer. A tube containing approximately 3 ml of water was also placed in the thermomixer along with a thermometer. The temperature was recorded after the increase in temperature plateaued and no longer increased. The characteristics of the wax in the reaction vessel was then recorded. The setting was then adjusted upward and the process repeated. Melting temperature (Tm) for each wax-oil combination was the temperatures at which wax was visibly melted in the reaction vessel and flowed freely. Several reaction vessels of each combination were made with approximately 3 ml of wax in each reaction vessel. Some reaction vessels were placed on ice to observe the behavior of the wax at the lower temperature and to test for seal integrity as measured by observed shrinkage from the reaction vessel walls. The 25% paraffin/heavy mineral oil combination was used for some of the subsequent experiments. Results are shown in Table 1.

TABLE 1
Paraffin wax-mineral oil compositions melted and cooled on ice.

| Wax | % Wax | Oil | Shrinkage | Tm |
|---|---|---|---|---|
| Paraffin 58 | 25% | Light | great | 46.3 |
| Paraffin 58 | 25% | Heavy | some | 51.3 |
| Paraffin 65 | 25% | Light | little | 55 |
| Paraffin 65 | 25% | Heavy | very little | 55 |
| SuperCera ® | 20% | Heavy | very little | 60.4 |
| Wax-Green | | | | |

Further similar tests were performed to determine melting behavior, melting temperature, and integrity of additional microcrystalline wax-mineral oil combinations. Castaldo SuperCera® Wax-Green (Castaldo, Staffordshire, UK) was mixed with appropriate quantities of heavy white mineral oil (Mineral Oil White, heavy; Avantor Performance Materials, Radnor, PA) to prepare 5%, 7.5%, 10%, 20%, and 25% wax solutions by weight and were heated at 75-80° C. To test melting behavior, three tubes of each percentage were prepared, with 2.5 ml wax solution per tube. All tubes were placed in a heat block and the heat block temperature was gradually increased. Melting behavior and temperatures are shown in Table 2. No change in wax composition appearance was recorded as “solid.” If the wax composition cleared but did not flow, it was recorded as “soft.” If the wax composition flowed only very slowly, it was recorded as “viscous.” If the wax composition flowed freely, it was recorded as “melted.” All tubes were then placed at 4ºC for several minutes until solidified; none of the solutions were observed to pull away from the sides of the tubes.

TABLE 2
Microcrystalline wax-mineral oil compositions melted and cooled

% Wax in Heavy Mineral Oil

| Temp | 5% | 7.5% | 10% | 20% | 25% |
|---|---|---|---|---|---|
| 30.3 | viscous | soft | soft | solid | solid |
| 33 | viscous | soft | soft | solid | solid |
| 37.9 | viscous | soft | soft | solid | solid |
| 40.3 | viscous | viscous | soft | solid | solid |
| 44.8 | viscous | viscous | soft | solid | solid |
| 48.6 | viscous | viscous | viscous | solid | solid |
| 53.2 | melted | melted | viscous | solid | solid |
| 57.6 | melted | melted | viscous | solid | solid |
| 61.5 | melted | melted | viscous | soft | solid |

### Displacement Tests

For a more representative test of melting temperature, and to more accurately mimic the interaction between solubilized lysis paste and wax layers of the intermediate layer, displacement tests were performed for a range of wax compositions. A layer of lysis buffer (4.7 M GITC in water) was added on top of the wax, and the temperature at which the lysis buffer layer would sink below the softened and melted wax layer was determined. Reaction vessels were prepared with 3.25 ml wax composition per vessel, with 0.8 ml lysis buffer added above the solidified wax in each vessel. Results are shown in Tables 3 and 4. If the lysis buffer remained on top of the 10 melted wax layer, it was scored as “float”; if the lysis buffer sank below the melted wax layer it was scored as “sink.”

TABLE 3
Displacement tests

SuperCera ® Wax-Green in Heavy Mineral Oil

| Temp | 10% | 20% | 25% | 30% |
|---|---|---|---|---|
| 60.6 | Sink | Float | Float | Float |
| 63.1 | Sink | Float | Float | Float |
| 65.3 | Sink | Float | Float | Float |
| 66.3 | Sink | Float | Float | Float |
| 67.3 | Sink | Sink | Sink | Float |

Repeat Study

| Temp | 18% | 20% | 25% |
|---|---|---|---|
| 60 | Float | Float | Float |
| 63.3 | Float | Float | Float |
| 64.5 | Sink | Float | Float |
| 65.3 | Sink | Sink | Float |
| 66.2 | Sink | Sink | Float |
| 67.3 | Sink | Sink | Sink |

To test the integrity of SuperCera® green wax/heavy mineral oil solutions, reaction vessels were prepared with alternating layers of 5% and 18% wax solutions, as shown in FIG. 1D. The vessels were placed at 4° C. for several minutes until solidified and heated to 60° C. and permitted to cool. No shrinkage was observed at 4° C., and no leaking or gaps were observed following heating to 60° C. As shown in FIG. 1D, the lysis paste on top of the wax layers remained in place, and the pH strips embedded in agarose beneath the wax layers showed no changes. However, during storage of a small piece of the wax with lysis paste, some pigment leached out, indicating that the green pigment in the SuperCera® wax could be problematic for long-term storage with the concentrated lysis buffer. Therefore, further work and experimentation was restricted to paraffin wax compositions.

Results of displacement tests with paraffin wax/mineral oil, carnuba wax/mineral oil, and paraffin wax/carnuba wax/mineral oil compositions are shown in Table 4. These displacement tests were performed as described herein above for SuperCera® wax-green/mineral oil compositions. Carnuba wax compositions were evaluated as an alternative to microcrystalline wax (SuperCera® wax-green) and were tested to determine whether the amount of shrinkage was different than that of the paraffin wax, but were not pursued further due to their high melting temperatures.

TABLE 4
Displacement tests

| | | | 5% Carnuba Wax |
|---|---|---|---|
| Paraffin 65 wax in Heavy Mineral | | | in Heavy Mineral |
| Oil | | | Oil |
| Temperature | 5% P | 25% P | 5% C |
| 31.8 | Float | Float | Float |
| 37.5 | Float | Float | Float |
| 42.2 | Float | Float | Float |
| 47.4 | Sink | Float | Float |
| 49.3 | Sink | Float | Float |
| 52.3 | Sink | Float | Float |
| 57.4 | Sink | Sink | Float |
| 62.5 | Sink | Sink | Float |
| 67.6 | Sink | Sink | Float |
| 72.6 | Sink | Sink | Float |

| | 15% Paraffin-1% |
|---|---|
| | Carnuba Wax in Heavy |
| | Mineral Oil |
| Temperature | 15% P-1% C |
| 47.5 | Float |
| 52.6 | Float |
| 55.5 | Float |
| 56.4 | Float |
| 57.5 | Float |
| 58.4 | Float |
| 59.4 | Float |
| 60.2 | Float |
| 61.6 | Float |
| 62.2 | Sink |

Repeat Study

| Temperature | 5% P | 25% P | 12% P-0.8% C |
|---|---|---|---|
| 28 | Float | Float | Float |
| 32.8 | Float | Float | Float |
| 37.5 | Float | Float | Float |
| 40.6 | Float | Float | Float |
| 43.7 | Sink | Float | Float |
| 47.6 | Sink | Float | Float |
| 52.5 | Sink | Float | Float |
| 55.5 | Sink | Float | Float |
| 57.4 | Sink | Sink | Float |
| 59.2 | Sink | Sink | Float |
| 61.1 | Sink | Sink | Float |
| 63.2 | Sink | Sink | Float |
| 65.1 | Sink | Sink | Float |
| 66.1 | Sink | Sink | Float |
| 66.8 | Sink | Sink | Sink |

The overall results of wax composition testing are summarized in Table 5.

TABLE 5

| | | | | | | 15% Paraffin-1% | |
|---|---|---|---|---|---|---|---|
| | SuperCera ® wax- | | | | | Carnuba Wax in Heavy | |
| | green in Heavy | | | Paraffin 65 wax in | | Mineral Oil | |
| Temper- | Mineral Oil | | | Heavy Mineral Oil | | 15% P- | 12% P- |
| ature | 18% | 20% | 25% | 5% P | 25% P | 1% C | 0.8% C |
| 47.4 | | | | Sink | | | |
| 57.4 | | | | | Sink | | |
| 62.2 | | | | | | Sink | |
| 64.5 | Sink | | | | | | |
| 65.3 | | Sink | | | | | |
| 66.8 | | | | | | | Sink |
| 67.3 | | | Sink | | | | |

For integrity testing of 25% paraffin/75% mineral oil and 5% paraffin/95% mineral oil layers, reaction vessels were prepared as follows and as shown in FIG. 2B. 3.5 ml of 0.7% agarose was prepared in System Diluent (Abbott, Abbott Park, Ill.) and melted on a hot plate. Reaction vessel bottoms were capped with Alinity m reaction tube bottoms (50 μl; Abbott, Abbott Park, Ill.). Indicator strips were placed into reaction vessels; agarose was added and let solidify. Wax layers were added sequentially above the agarose layer, alternating 0.3 ml low-temperature meltable wax (25% paraffin/75% mineral oil) with 0.3 ml lower-temperature meltable wax (5% paraffin/95% mineral oil) as shown in FIG. 2B. Each wax layer was allowed to solidify before the next was added. Finally, 0.5 g lysis paste containing CuTi particles was layered on top of the last wax layer. Reaction vessels were stored at room temperature for one year and examined periodically for wax shrinkage and leakage. No shrinkage or leakage was observed, indicating that the 5% paraffin wax layer did not shrink from the reaction vessel walls, and indicating that it can act to seal the lysis buffer from the aqueous agarose layer.

### Extraction Tube Assembly and Testing

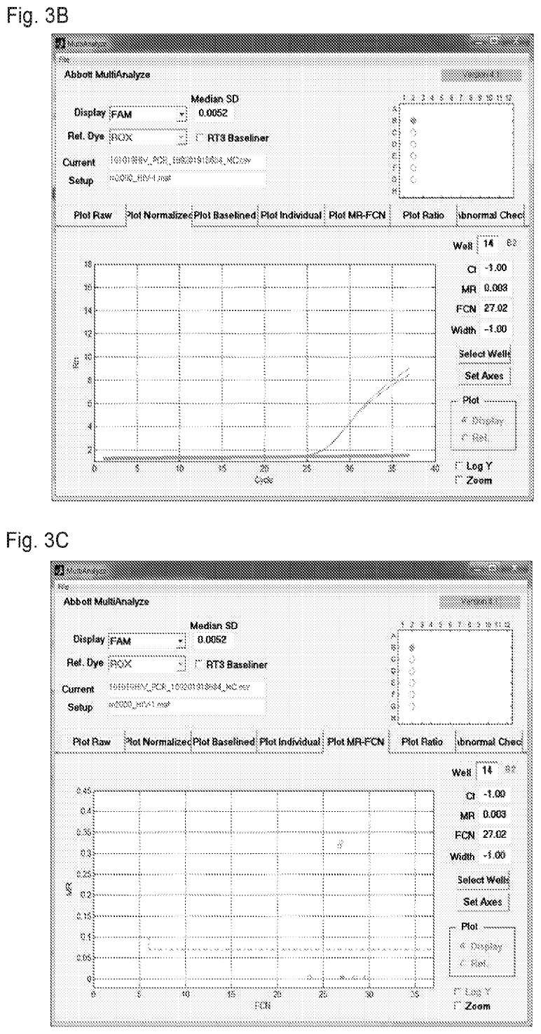

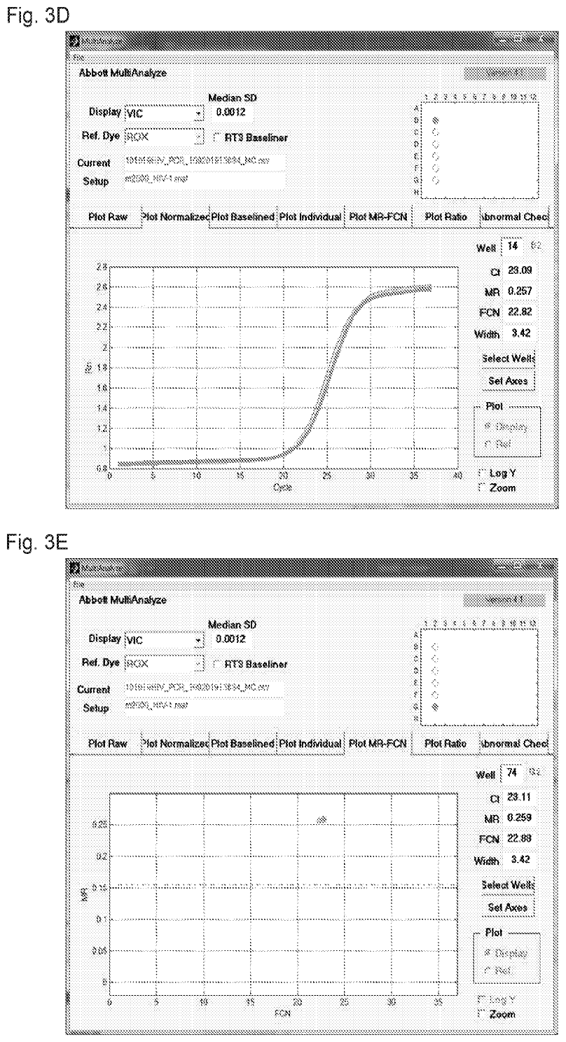

Basic extraction tube designs were prepared to test the melting process, using 5 ml pipette tips (VWR, Radnor, PA) as extraction tubes. The tubes were prepared according to the schematic diagram shown in FIG. 3A, with two wax layers separated by an agarose layer. A melted 25% wax solution (25% paraffin/75% Chill-out™ liquid wax) was prepared and held at approximately 75° C. 100 ml of 0.5% agarose was prepared in System Diluent (Abbott, Abbott Park, Ill.) by heating to boiling and allowed to cool but not solidify. While the agarose cooled, the lower tip of the extraction tube was sealed with an Alinity m reaction vessel bottom (Abbott, Abbott Park, Ill.). The cooled but still liquid agarose was then added to the extraction tube, forming an aqueous gel layer, and allowed to solidify. Next, 0.4 ml cooled 25% wax was added, forming a wax layer above the agarose gel layer. After the 25% wax solidified, 0.4 ml cooled 0.5% agarose was added and allowed to solidify, forming an agarose gel layer above the 25% wax layer. 0.3 ml 25% wax was then added above the second agarose gel layer and allowed to solidify. 17 μl of HIV internal control (IC) was added, followed by another 0.3 ml 25% wax. Finally, 0.5 g lysis paste containing CuTi particles was added above the final solidified wax layer.

Heating blocks with holes that open on both ends were held at 75-80° C. Blocks were set up such that the undersides of the blocks were exposed. Tubes were placed into the blocks such that the lower agarose layers stuck out of the undersides of the blocks, allowing that region to stay cooler than the heated upper regions.

As an initial test sample, 0.5 ml water was added to the lysis paste, dissolving the lysis paste and releasing the bubbles within the paste. The tubes were then placed into a heating block for several minutes to determine whether the intermediate wax layers melted. Melting was complete within five minutes as described below herein. The wax layers melted sequentially, with the top layer melting first, then the second. Each time, the wax floated to the top of the lysis paste layer, mixing the lysis paste-sample-particles mix and removing a need for mechanical mixing. When cooled, the wax sealed the top of the tube so no liquid waste from the sample. The lower agarose gel layer did not melt and remained in the tube, sealing the bottom.

### Extraction System Testing

Experiments were performed to test the simple extraction system. Six extraction tubes were prepared according to the schematic diagram shown in FIG. 3A and as described previously herein. Of the 6 samples tested, two were HIV samples and four were control samples (base matrix (purified human plasma) without HIV). A melted 25% wax solution (25% paraffin/75% Chill-out™ liquid wax) was prepared and held at approximately 75° C. 1 ml of 0.5% agarose was prepared and allowed to cool but not harden. While the agarose cooled, the lower tip of the extraction tube was sealed with an Alinity m reaction vessel bottom (Abbott, Abbott Park, Ill.). The cooled agarose was then added to the extraction tube, forming an aqueous gel layer, and allowed to solidify. Next, 0.4 ml cooled 25% wax was added, forming a wax layer above the agarose gel layer. After the 25% wax solidified, 0.4 ml cooled but still liquid agarose was added and allowed to solidify, forming an agarose gel layer above the 25% wax layer. Another 25% wax layer (0.3 ml) was then added above the second agarose gel layer and allowed to solidify. 17 μl of HIV internal control (IC) was added, followed by another 0.3 ml 25% wax layer. Finally, 0.5 g lysis paste was added above the final solidified wax layer.

Sample extraction was performed as follows. A heat block was maintained at 75-80° C. A 0.5 ml sample was added to each extraction tube, and the tubes were placed in the heat block for five minutes, which was sufficient time to melt all wax layers. The heat block only heated the lysis reaction and wax layers of the tube; the lower agarose wash layer was not heated. Tubes were incubated for an additional 10-15 minutes in the heat block for the lysis reaction to proceed to completion. A magnetic force was applied to capture the CuTi particles within the lysate, pull the particles to the side of the tube, and through the aqueous agarose gel layer. The lower tip of the extraction tube was unsealed, the extraction tube was placed into an empty 200 μl MicroAmp tube (Applied Biosystems, Waltham, MA), and the CuTi particles were magnetically pulled into the microamp tube. To elute the nucleic acids bound to the CuTi particles, 100 μl elution buffer was added to the microamp tube, and the reaction was incubated at 75ºC for 10 minutes without mixing. The particles were then magnetically captured, and 50 μl of eluate was loaded into an HIV RT-PCR assay.

The eluates were tested with an HIV RT-PCR assay to determine whether HIV RNA was successfully extracted from the samples (Program 0.6 ml HIV-1 RNA version 7.00, Alinity m2000RT PCR System, Abbott, Abbott Park, Ill.). To each polymerase bottle, 271 μl activator and 941 μl oligo mix were added. To each amplification plate well, 50 μl master mix and 50 μl sample were added. As shown in FIGS. 3B and C, HIV RNA was successfully extracted from the two positive samples, and as shown in FIGS. 3D and E, the internal control worked in all six samples. These results demonstrated that the extraction system worked, and that an internal control could be successfully embedded in the wax layers and recovered during the extraction process.

A second set of tests were performed using the same reagents and under the same conditions, using 24 tubes. Extraction tubes were prepared as previously described herein and as shown in FIG. 3A. Four heating blocks were maintained at 75-80° C., and six tubes were incubated per block. Samples used for extraction are shown in Table 6.

TABLE 6
Samples used
Samples

| | | |
|---|---|---|
| 1 to 3 | Base Matrix | Purified |
| | | Human Plasma |
| 4 to 6 | HIV Cal A | HIV RNA |
| | Abbott 2G31A4 | synthetic target |
| | “Cal A” | in Base Matrix |
| 7 to 9 | HIV Cal B | HIV RNA |
| | Abbott 2G31B | synthetic target |
| | “Cal B” | in Base Matrix |
| 10 to 12 | HIV Control L | HIV RNA |
| | Abbott 2G31W | synthetic target |
| | “LoPos” | in Base Matrix |
| 13 to 15 | HIV Control H | HIV RNA |
| | Abbott 2G31X | synthetic target |
| | “HiPos” | in Base Matrix |
| 16 to 24 | 10X LOD | Inactive HIV |
| | (~200 copies | virion in Base |
| | per ml) | Matrix |
| | “10X LOD” | |

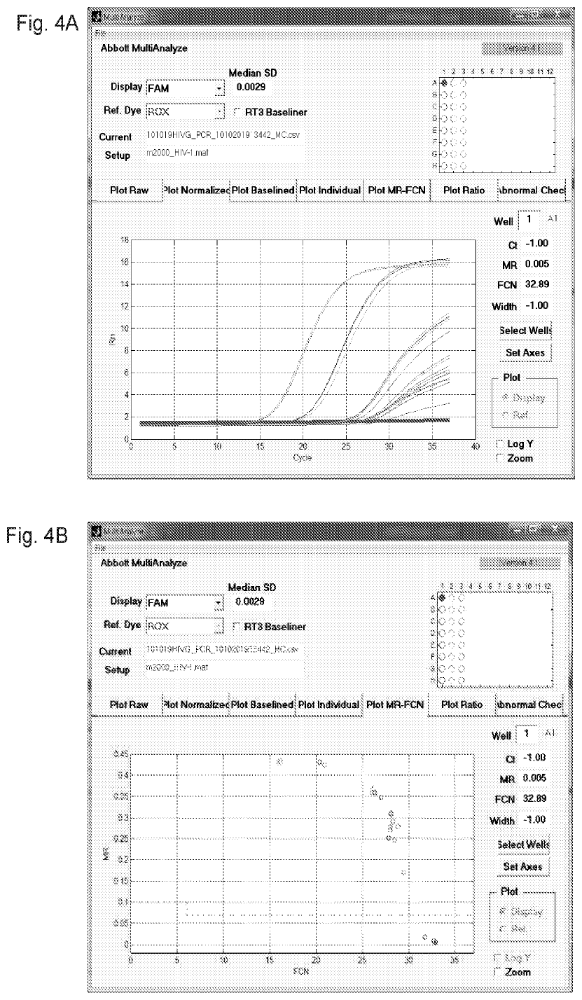

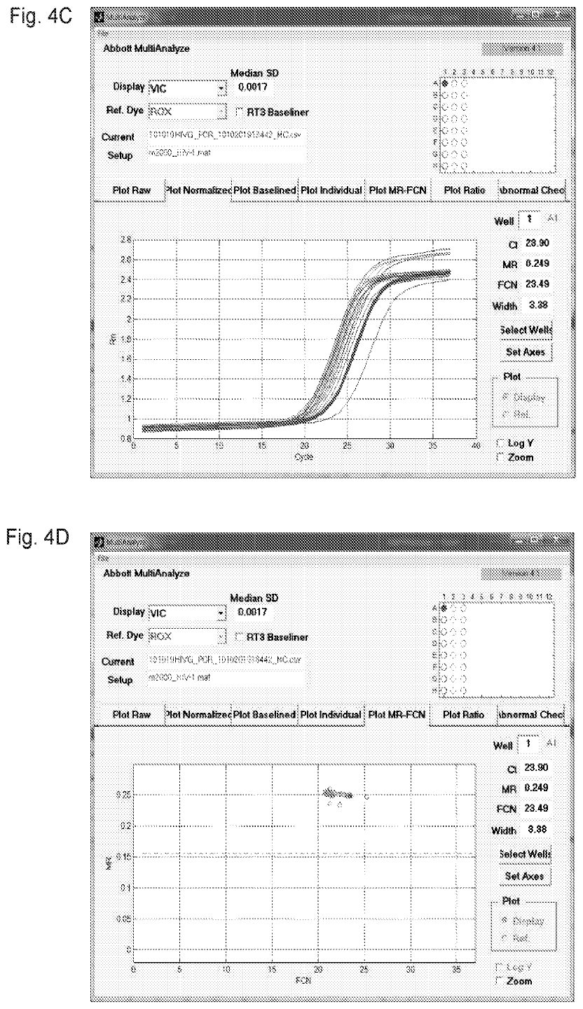

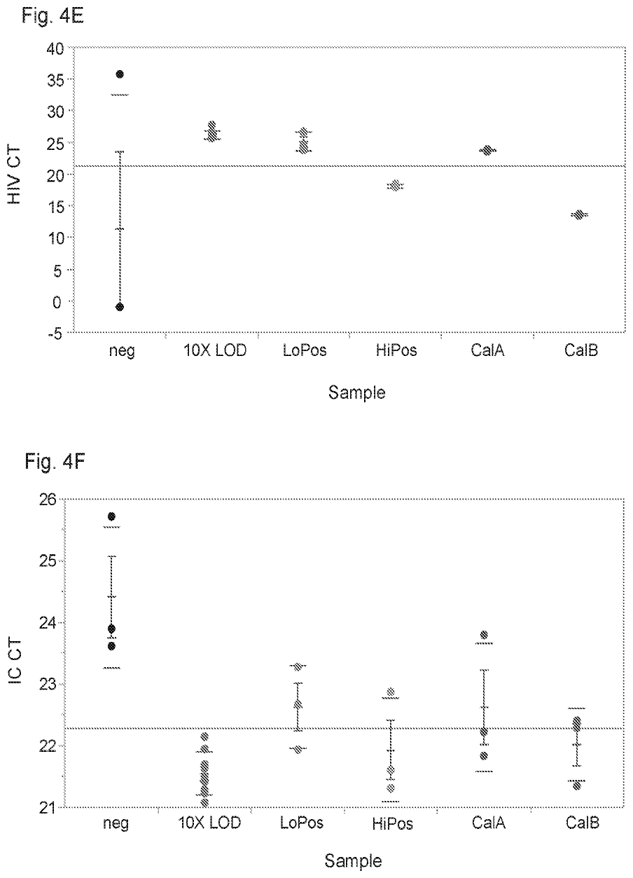

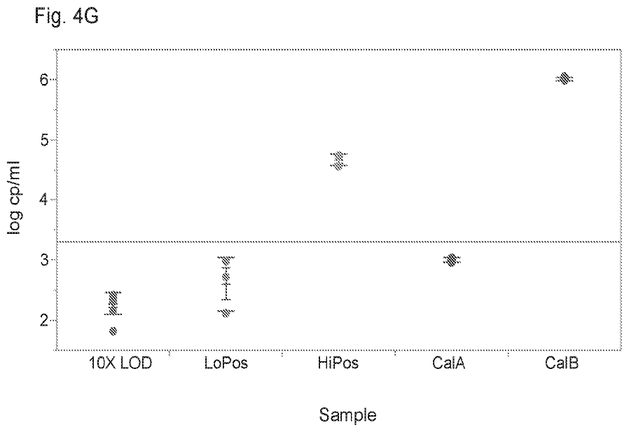

As previously described herein, the CuTi particles within the lysate were magnetically captured and pulled through the aqueous agarose gel layer into MicroAmp tubes. Nucleic acids were eluted from the CuTi particles using 75 μl elution buffer and a 10 minute incubation at 75° C. The CuTi particles were magnetically captured and 50 μl of each eluate was transferred to the assay plate after the rest of the assay reagents were loaded. Assay samples were arranged as shown in Table 7. As shown in FIGS. 4A-G and Tables 7-10, the nucleic acid extractions were successful.

TABLE 7

| | | | | HIV | | |
|---|---|---|---|---|---|---|
| | | | | CT | IC CT | Conc. |
| neg | HIV_NEG | A1 | 1 | −1 | 23.9 | |
| HiPos | HP | A2 | 2 | 18.45 | 21.31 | 4.56 |
| 10X | 2 | A3 | 3 | 25.68 | 21.95 | 2.42 |
| LOD | | | | | | |
| neg | NEG | B1 | 13 | 35.77 | 25.72 | |
| CalA | HIV_CALA | B2 | 14 | 23.86 | 22.23 | 2.96 |
| 10X | 3 | B3 | 15 | 26.03 | 21.23 | 2.32 |
| LOD | | | | | | |
| neg | NEG | C1 | 25 | −1 | 23.62 | |
| CalA | HIV_CALA | C2 | 26 | 23.58 | 21.84 | 3.04 |
| 10X | 4 | C3 | 27 | 25.8 | 21.29 | 2.39 |
| LOD | | | | | | |
| LoPos | HIV_LOPOS | D1 | 37 | 24.67 | 22.67 | 2.72 |
| CalA | HIV_CALA | D2 | 38 | 23.73 | 23.8 | 3 |
| 10X | 5 | D3 | 39 | 26.6 | 21.42 | 2.15 |
| LOD | | | | | | |
| LoPos | LP | E1 | 49 | 26.69 | 23.28 | 2.12 |
| CalB | HIV_CALB | E2 | 50 | 13.43 | 21.35 | 6.05 |
| 10X | 6 | E3 | 51 | 25.88 | 22.15 | 2.36 |
| LOD | | | | | | |
| LoPos | LP | F1 | 61 | 23.81 | 21.94 | 2.98 |
| CalB | HIV_CALB | F2 | 62 | 13.67 | 22.41 | 5.98 |
| 10X | 7 | F3 | 63 | 27.72 | 21.5 | 1.82 |
| LOD | | | | | | |
| HiPos | HIV_HIPOS | G1 | 73 | 17.93 | 22.88 | 4.72 |
| CalB | HIV_CALB | G2 | 74 | 13.59 | 22.29 | 6 |
| 10X | 8 | G3 | 75 | 25.97 | 21.08 | 2.34 |
| LOD | | | | | | |
| HiPos | HP | H1 | 85 | 17.86 | 21.61 | 4.74 |
| 10X | 1 | H2 | 86 | 25.86 | 21.7 | 2.37 |
| LOD | | | | | | |
| 10X | 9 | H3 | 87 | 25.89 | 21.64 | 2.36 |
| LOD | | | | | | |

TABLE 8
Oneway analysis of HIV CT by sample
(means and standard deviations)

| | | | | Std Err | Lower | Upper |
|---|---|---|---|---|---|---|
| Level | Number | Mean | Std Dev | Mean | 95% | 95% |
| neg | 3 | 11.2567 | 21.2292 | 12.257 | −41.48 | 63.993 |
| 10X | 9 | 26.1589 | 0.6405 | 0.213 | 25.67 | 26.651 |
| LOD | | | | | | |
| LoPos | 3 | 25.0567 | 1.4784 | 0.854 | 21.38 | 28.729 |
| HiPos | 3 | 18.0800 | 0.3223 | 0.186 | 17.28 | 18.881 |
| CalA | 3 | 23.7233 | 0.1401 | 0.081 | 23.38 | 24.071 |
| CalB | 3 | 13.5633 | 0.1222 | 0.071 | 13.26 | 13.867 |

TABLE 9
Oneway analysis of internal control CT by sample
(means and standard deviations)

| | | | | Std Err | Lower | Upper |
|---|---|---|---|---|---|---|
| Level | Number | Mean | Std Dev | Mean | 95% | 95% |
| neg | 3 | 24.4133 | 1.14023 | 0.65831 | 21.581 | 27.246 |
| 10X | 9 | 21.5511 | 0.34657 | 0.11552 | 21.285 | 21.818 |
| LOD | | | | | | |
| LoPos | 3 | 22.6300 | 0.67089 | 0.38734 | 20.963 | 24.297 |
| HiPos | 3 | 21.9333 | 0.83345 | 0.48119 | 19.863 | 24.004 |
| CalA | 3 | 22.6233 | 1.03751 | 0.59901 | 20.046 | 25.201 |
| CalB | 3 | 22.0167 | 0.58046 | 0.33513 | 20.575 | 23.459 |

TABLE 10
Oneway analysis of log cp/ml by sample
(means and standard deviations)

| | | | | Std Err | Lower | Upper |
|---|---|---|---|---|---|---|
| Level | Number | Mean | Std Dev | Mean | 95% | 95% |
| 10X | 9 | 2.28111 | 0.189172 | 0.06306 | 2.1357 | 2.4265 |
| LOD | | | | | | |
| LoPos | 3 | 2.60667 | 0.441059 | 0.25465 | 1.5110 | 3.7023 |
| HiPos | 3 | 4.67333 | 0.098658 | 0.05696 | 4.4283 | 4.9184 |
| CalA | 3 | 3.00000 | 0.040000 | 0.02309 | 2.9006 | 3.0994 |
| CalB | 3 | 6.01000 | 0.036056 | 0.02082 | 5.9204 | 6.0996 |

### Example 3. Additional Testing and Verification of Nucleic Acid Extraction System Components and Methods

### Materials and Methods

Materials and methods used were as described in Examples 1 and 2, except as described herein below.

### Extraction System Testing

Additional experiments were performed to test embodiments of the simple extraction system in which the lower-temperature meltable wax layers served as intervening hydrophobic sealing layers. Two sets of 5 ml pipette tip reaction vessels were prepared according to the schematic diagrams shown in FIG. 5A, one set with an intermediate layer of five alternating low- and lower-temperature meltable wax layers (“five-layer”) and one set with an intermediate layer of six alternating low- and lower-temperature meltable wax layers (“six-layer”). Two wax solutions were prepared and held at approximately 80-85° C., one was a low-temperature melting 15% wax solution (15% paraffin/85% mineral oil), and the other was a lower-temperature melting 5% wax solution (5% paraffin/95% mineral oil). 0.5% agarose was prepared and held at 80-85° C. until reaction vessel preparation. The lower tip of the reaction vessel was sealed with an Alinity m reaction vessel bottom (Abbott, Abbott Park, Ill.); and 2 ml of 0.5% agarose was added to the reaction vessels and allowed to cool and harden, forming an aqueous gel wash layer.

As illustrated in FIG. 5A, the set of five-layer reaction vessels was prepared by adding alternating layers of low-temperature melting 15% and lower-temperature melting 5% (intervening hydrophobic sealing layers) wax solutions above the gel wash layer. Each wax layer was allowed to solidify at room temperature before the subsequent wax layer was added; 0.3 ml wax was used for the first four layers, and 0.4 ml wax was used for the final layer. As additionally illustrated in FIG. 5A, the set of six-layer reaction vessels was prepared by adding alternating layers of lower-temperature melting 5% (intervening hydrophobic sealing layers) and low-temperature melting 15% wax solutions above the gel wash layer. Each wax layer was allowed to solidify before the subsequent wax layer was added; 0.3 ml wax was used for the first four layers, and 0.4 ml wax was used for the final layer. Finally, 0.5 g lysis paste with CuTi particles was added above the final solidified wax layer.

Four samples were tested for each set, two negative control samples (“Neg”; HIV-1 Neg Control 2G31Z (Abbott, Abbott Park, Ill.) and HIV internal control (IC)), and two positive control samples (“Pos”; HIV-Hi Pos Control 2G31X (Abbott, Abbott Park, Ill.) and HIV internal control (IC)). Sample extraction was performed as follows. A heat block was maintained in a laboratory oven (Cole-Parmer, Vernon Hills, IL) at 80° C. For both five- and six-layer reaction vessels, 17 μl HIV internal control (IC) was added to the lysis paste, and 0.5 ml of the respective sample was added to the lysis paste in each reaction vessel. Extractions were performed two at a time; two reaction vessels were placed in the heat block in the laboratory oven. With this arrangement all layers in the reaction vessels were subjected to substantially uniform heating. For both five- and six-layer reaction vessels, the wax layers appeared to be melted completely after approximately three minutes and the lysis reaction with the particles had sunk below the wax layers and was in fluid communication with the gel wash layer. After five minutes, a magnetic force was applied to capture the CuTi particles within the lysis reaction, pull the particles to the side of the tube and through the agarose gel wash layer. The lower tip of the reaction vessel was unsealed and placed into a 200 μl MicroAmp tube (Applied Biosystems, Waltham, MA) containing 100 μl elution buffer, and the CuTi particles were magnetically pulled into the MicroAmp tube. To elute the nucleic acids bound to the CuTi particles, the reaction was incubated at 75ºC for five minutes without mixing. The particles were then magnetically captured, and 50 μl of eluate was loaded into an HIV RT-PCR assay.

The eluates were tested with an HIV RT-PCR assay to determine whether HIV RNA was successfully extracted from the samples (Program 0.6 ml HIV-1 RNA version 8.00, Alinity m2000RT PCR System, Abbott, Abbott Park, Ill.). To each polymerase bottle, 271 μl activator and 941 μl oligo mix were added. To each amplification plate well, 50 μl master mix and 50 μl sample were added.

### Results

For both sets of extractions, as shown in FIG. 5B-C and Table 11, HIV RNA was successfully extracted from the positive samples (Target CT column), and the internal control worked in all samples (IC CT column).

Table 11. Results for five- and six-layer extractions

TABLE 11
Results for five- and six-layer extractions

| | | Target | |
|---|---|---|---|
| Tube | Sample | CT | IC CT |
| 5 layer | Neg | −1 | 31.92 |
| 5 layer | Neg | −1 | 31.00 |
| 5 layer | Pos | 27.70 | 28.48 |
| 5 layer | Pos | 26.35 | 31.77 |
| 6 Layer | Neg | −1 | 27.73 |
| 6 Layer | Neg | −1 | 29.38 |
| 6 Layer | Pos | 26.27 | 27.53 |
| 6 Layer | Pos | 27.40 | 28.32 |

Embodiments of the simple extraction system have been demonstrated to work using an HIV assay and HIV sample controls. Samples were successfully and efficiently extracted using both five- and six-layer embodiments with intermediate layers comprising intervening sealing layers of lower-temperature meltable wax. No meaningful differences were observed between the embodiments.

### Example 4. Testing Nucleic Acid Extraction System Components and Methods for Isolation of HBV DNA

To isolate hepatitis B virus (HBV) DNA, concentrated lysis solution was loaded into the meltable wax-based, multi-layered extraction cartridges of the present invention, and re-hydrated by the sample. Using CuTi particles on the Alinity m automated instrument, HBV DNA was isolated without the need to pump lysis solution, thereby reducing complexity of the automated platform. These data show that embodiments of the extraction system have been demonstrated to work using an HBV assay and HBV sample controls.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1) A multi-layered composition for the extraction and isolation of nucleic acids from a biological sample, the multi-layered composition comprising:
a) a reaction vessel in which layers of the multi-layered composition are assembled;
b) an uppermost layer within the reaction vessel comprising a concentrated semi-solid lysis paste;
c) an intermediate layer comprising a low-temperature meltable wax; and
d) a lower wash layer comprising an aqueous gel.

2) The multi-layered composition of claim 1 wherein the reaction vessel comprises a tube of substantially circular cross-section, the tube having a top and a bottom.

3) The multi-layered composition of claim 2 wherein the top and the bottom of the tube are reversibly sealed.

4) The multi-layered composition of claim 2 wherein the tube is J-shaped or U-shaped.

5) The multi-layered composition of claim 1 wherein the biological sample is a liquid biological sample.

6) The multi-layered composition of claim 5 wherein the liquid biological sample is a serum sample, a blood sample, a plasma sample, a saliva sample, or other type of biological sample.

7) The multi-layered composition of claim 1 wherein the low-temperature meltable wax is solid at room temperature.

8) The multi-layered composition of claim 1 wherein the low-temperature meltable wax begins to transition to liquid at a temperature above 50-55 degrees C.

9) The multi-layered composition of claim 1 further comprising an adjacent layer comprising an elution buffer in fluid communication with the wash layer.

10) The multi-layered composition of claim 9 wherein the elution buffer is a low ionic strength elution buffer.

11) The multi-layered composition of claim 10 wherein the low ionic strength elution buffer is a phosphate buffer.

12) The multi-layered composition of claim 1 wherein the intermediate layer comprises a plurality of low-temperature meltable wax layers wherein each of the plurality of low-temperature meltable wax layers is separated by an intervening sealing layer.

13) The multi-layered composition of claim 12 wherein the intervening sealing layer separating each of the plurality of low-temperature meltable wax layers comprises a lower-temperature meltable wax that melts at a lower temperature than the layer immediately below it.

14) The multi-layered composition of claim 12, wherein the intervening sealing layer separating each of the plurality of low-temperature meltable wax layers comprises mineral oil.

15) The multi-layered composition of any one of claims 12-14 wherein the plurality of low-temperature meltable wax layers are layered such that, in use, each of the plurality of low-temperature meltable wax layers melts sequentially from uppermost to lowermost.

16) The multi-layered composition of claim 1 wherein the intermediate layer further comprises an internal control.

17) The multi-layered composition of claim 1 wherein the concentrated semi-solid lysis paste is comprised of GITC, Tris-HCl, Tris base, and Tween-20.

18) The multi-layered composition of claim 17 wherein the concentrated semi-solid lysis paste has a residual moisture of less than 10% and does not flow.

19) The multi-layered composition of claim 18 wherein the concentrated semi-solid lysis paste has a residual moisture of less than 5%.

20) The multi-layered composition of claim 1 wherein the aqueous gel is comprised of agarose or polyacrylamide.

21) The multi-layered composition of claim 31 wherein the plurality of metal oxide coated particles are present in a quantity expected to represent a molar excess relative to the quantity of nucleic acids calculated to be present in the biological sample.

22) A method for extracting and purifying nucleic acids from a biological sample, the method comprising:
a) providing a multi-layered composition for the extraction and isolation of nucleic acids from the biological sample, the multi-layered composition comprising:
i) a reaction vessel in which layers of the multi-layered composition are assembled;
ii) an uppermost layer within the reaction vessel comprising a concentrated semi-solid lysis paste;
iii) an intermediate layer comprising a low-temperature meltable wax; and
iv) a lower wash layer comprising an aqueous gel;
b) layering a biological sample above the uppermost layer thereby forming solubilized lysis paste, wherein the solubilized lysis paste further comprises a plurality of metal oxide coated magnetic particles;
c) heating the reaction vessel to initiate melting of the intermediate layer resulting in density inversion characterized by the melting intermediate layer and rising through the solubilized concentrated semi-solid paste thereby mixing the biological sample, the solubilized concentrated semi-solid lysis paste and the plurality of metal oxide coated magnetic particles thereby resulting in the binding of nucleic acids within the biological sample to the metal oxide coated particles;
d) positioning a magnet around or adjacent to the reaction vessel thereby attracting metal oxide coated particles and bound nucleic acids;
e) moving the magnet thereby driving the migration of attracted metal oxide coated particles and bound nucleic acids at least partially through the aqueous gel thereby removing extraction contaminants.

23) The method of claim 22 wherein the low-temperature meltable wax is solid at room temperature.

24) The method of claim 22 wherein the low-temperature meltable wax begins to transition to liquid at a temperature above 50-55 degrees C.

25) The method of claim 22 wherein the multi-layered composition further comprises an elution buffer in fluid communication with the wash layer, and the magnet is moved thereby driving the migration of attracted metal oxide coated particles and bound nucleic acids through the aqueous gel and into the elution buffer.

26) The method of claim 25 wherein the elution buffer is a low ionic strength elution buffer, optionally further wherein the low ionic strength elution buffer is a phosphate buffer.

27) The method of claim 22 wherein the intermediate layer of the multi-layered composition comprises a plurality of low-temperature meltable wax layers wherein each of the plurality of low-temperature meltable wax layers is separated by an intervening sealing layer.

28) The method of claim 27 wherein the intervening sealing layer of the multi-layered composition separating each of the plurality of low-temperature meltable wax layers comprises a lower-temperature meltable wax that melts at a lower temperature than the layer immediately below it.

29) The method of claim 27 wherein the intervening sealing layer of the multi-layered composition separating each of the plurality of low-temperature meltable wax layers comprises mineral oil.

30) The method of claim 27 wherein the plurality of low-temperature meltable wax layers are layered such that, in use, each of the plurality of low-temperature meltable wax layers melts sequentially from uppermost to lowermost as the reaction vessel is heated.

31) The multi-layered composition of claim 1 wherein the uppermost layer of element b) further comprises a plurality of metal oxide coated magnetic particles.

32) The multi-layered composition of claim 31 wherein the metal oxide coated particles are titanium oxide particles.

33) The multi-layered composition of claim 31 wherein the titanium oxide coated particles are copper titanium (CuTi) coated particles.

34) The method of claim 22 wherein the plurality of metal oxide coated particles are titanium oxide particles.

35) The method of claim 34 wherein the titanium oxide particles are copper titanium (CuTi) coated particles.

36) The method of claim 22, wherein the plurality of metal oxide coated particles are provided following the provision of the multi-layered composition of element a).

37) The multi-layered composition of claim 12 wherein the intervening sealing layer separating each of the plurality of low-temperature meltable wax layers comprises agarose.

38) The method of claim 27 wherein the intervening sealing layer of the multi-layered composition separating each of the plurality of low-temperature meltable wax layers comprises agarose.
